(19) United States
(12) Patent Application Publication
(10) Pub. No.: US 2019/0054087 A1
(43) Pub. Date: Feb. 21, 2019

(54) TREATMENT OF SMALL CELL LUNG CANCER WITH A PARP INHIBITOR

(71) Applicant: Medivation Technologies LLC, San Francisco, CA (US)

(72) Inventors: Ying FENG, Novato, CA (US); Leonard E. POST, Novato, CA (US); Yuqiao SHEN, Novato, CA (US); Yuanbin RU, Novato, CA (US); Evelyn WANG, Novato, CA (US); Karen YU, Novato, CA (US)

(73) Assignee: Medivation Technologies LLC, San Francisco, CA (US)

(21) Appl. No.: 15/771,086

(22) PCT Filed: Oct. 26, 2016
(86) PCT No.: PCT/US16/58928

§ 371 Date: Apr. 25, 2018

Related U.S. Application Data
(60) Provisional application No. 62/246,538, filed on Oct. 26, 2015.

(51) Int. Cl. A61K 31/5025; A61P 35/00; G01N 33/574; C12Q 1/6886; A61K 9/00; A61K 45/06

(52) U.S. Cl. CPC A61K 31/5025; A61P 35/00; G01N 33/57423; C12Q 2600/106; A61K 9/0053; A61K 45/06; C12Q 2600/158; C12Q 1/6886

(57) ABSTRACT

Described are methods of treatment of a small cell lung cancer subject expressing Schlafen-11 (SLFN 11) with a Poly (ADP-ribose) polymerases (PARP) inhibitor or a pharmaceutically acceptable salt thereof. Specifically, the method comprising detecting SLFN 11 in a tumor cell sample from the subject, and administering effective amount of a PARP inhibitor, such as talazoparib or the tosylate salt of talazoparib, to the subject.

### CROSS-REFERENCE TO RELATED APPLICATIONS

This application claims priority from U.S. provisional application No. 62/246,538 filed Oct. 26, 2015, entitled “Treatment of Small Cell Lung Cancer with a PARP Inhibitor,” which is incorporated by reference in its entirety.

### FIELD OF THE INVENTION

Described herein are methods of treatment of a small cell lung cancer subject expressing Schlafen-11 (SLFN11) with a PARP inhibitor or with talazoparib or a pharmaceutically acceptable salt thereof.

### BACKGROUND OF THE INVENTION

Small cell lung cancer (SCLC) is an aggressive subtype of lung cancer, accounting for approximately 15% of all lung cancer cases in United States. SCLC is characterized by small cells with poorly defined cell borders and minimal cytoplasm, rare nucleoli, and finely granular chromatin. Due to the aggressive nature of the disease, the low rate of early diagnosis, and the lack of effective therapies, prognosis is generally poor. Median survival time from diagnosis for untreated SCLC patients is only two to four months. When chemotherapy and/or radiation modalities are used, the initial response rate to among SCLC patients is high (approximately 60 to 80%), but relapse occurs in the majority treated patients, who then are largely refractory to further systemic therapy. Thus, even with current treatment modalities, the median survival time for patients with limited-stage disease is 16 to 24 months and for patients with extensive disease, seven to 12 months. To improve patient survival rates, it is essential to treat patients with chemotherapeutic agents to which their tumors are sensitive. Use of targeted drugs in the treatment of SCLC represents a major unmet medical need. Unlike non-small cell lung cancers (NSCLC), there are currently no targeted therapies with demonstrated benefit for patients with this disease. Thus, there is a need to align SCLC patients with suitable treatments based on their individual genetic profiles. Understanding a given tumor's genetic profile will also enable early diagnosis, detection, and treatment selection.

Increased SLFN11 expression has been reported to correlate positively with increased sensitivity of SCLC cells to topoisomerase inhibitors, alkylating agents, and DNA-damaging agents. See, Zoppoli et al., PNAS USA 2012, 109(37), 15030-15035; Zoppoli et al., Cancer Res. 2012, 72(8 Supplement): 4693. Poly(ADP-ribose)polymerases (PARP) inhibitors are a more recent addition to the anti-cancer arsenal. Certain PARP inhibitors work as catalytic inhibitors as well as PARP poisons by trapping PARP-DNA complexes (Murai et al., Cancer Res. 2012: 72:5588-99). Talazoparib (BMN 673) is the most potent PARP inhibitor reported to date in terms of tumor cytotoxicity and PARP trapping activities (Shen et al., Clin. Cancer Res. 2013:19:5003-15; Murai et al., Mol. Cancer Ther. 2014:13:433-43). Talazoparib has demonstrated significant clinical activity in ovarian and breast cancer patients with deleterious germline BRCA1/2 mutations (De Bono et al., ASCO 2013, Abstract 2580). However, BRCA mutations have not been shown to predict greater sensitivity of SCLC to PARP inhibitors. Antitumor responses to talazoparib were also reported in SCLC patients (Wainberg et al., ASCO 2014, Abstract 7522). Previous studies identified a slate of DNA repair protein markers that correlates with talazoparib sensitivity in SCLC (Cardnell et al., Clin. Cancer Res. 2013, 19(22), 6322-6328), but these have not been validated clinically. In addition, an in vitro screen in the NCI60 cell line panel revealed that increased expression of Schlafen 11 (SLFN11) is correlated with increased cellular sensitivity to talazoparib exposure in a range of tumor cell lines (Murai et al., AACR 2014, Abstract 1718). However, the NCI60 panel lacks any SCLC-derived cell lines, so the question of whether any particular genetic features correlated with increased sensitivity of SCLC cells to PARP inhibitors or talazoparib was left unanswered. In sum, there are no validated genetic profiles in SCLC patients that predict responsiveness to PARP inhibitors. Identification of such determinants will allow for the identification of patients who may respond well to a PARP inhibitor, or to talazoparib in particular.

There remains a need for methods of treatment of certain genetically-sensitive SCLC patients with PARP inhibitors. Further, identifying validated, clinically relevant biomarkers for SCLC will allow for earlier detection and appropriate therapeutic targeting.

### BRIEF SUMMARY OF THE INVENTION

A study of a collection of 38 SCLC cell lines demonstrated that sensitivity to single-agent treatment of talazoparib correlates well with expression of each of the following genes: SLFN11, SIL1, SLC25A3, MAF, AP3B1, C1orf50, BCL2, DDX6, and GULP1. The in vitro sensitivity results were confirmed in vivo in several SCLC cell line-derived xenograft (CDX) models. Notably, an in vivo study using 12 patient-derived xenograft (PDX) samples of SCLC revealed a correlation between SLFN11expression (both at the messenger RNA level and at the protein level) and the sensitivity of the tumors to PARP inhibitor treatment.

Thus, the present invention includes methods of treating a SLFN11-, SIL1-, SLC25A3-, MAF-, AP3B1-, C1orf50-, BCL2-, DDX6-, and/or GULP1-positive SCLC patient comprising administering to the patient an effective amount of a PARP inhibitor. In another aspect, the present invention relates to a method of treating a SLFN11-positive SCLC patient comprising administering to the patient an effective amount of a PARP inhibitor. In other aspects, the invention relates to a method of treating a SLFN11-, SIL1-, SLC25A3-, MAF-, AP3B1-, C1orf50-, BCL2-, DDX6-, and/or GULP1-positive SCLC patient comprising administering to the patient an effective amount of talazoparib or a pharmaceutically acceptable salt thereof. In another aspect, the present invention relates to a method of treating a SLFN11-positive SCLC patient comprising administering to the patient an effective amount of talazoparib or a pharmaceutically acceptable salt thereof.

In one aspect, the invention relates to a method of treating SCLC in a subject expressing SLFN11, comprising administering to the subject an effective amount of a PARP inhibitor. In another aspect, the invention relates to a method of treating SCLC in a subject expressing SLFN11, comprising administering to the subject an effective amount of talazoparib or a pharmaceutically acceptable salt thereof.

In another aspect, the invention relates to a method of treating SCLC in a subject expressing one or more of SLFN11, SIL1, SLC25A3, MAF, AP3B1, C1orf50, BCL2, DDX6, or GULP1, comprising administering to the subject an effective amount of a PARP inhibitor, or an effective amount of talazoparib or a pharmaceutically acceptable salt thereof. In some aspects, the subject expresses SLFN11, and optionally expresses one or more of SIL1, SLC25A3, MAF, AP3B1, C1orf50, BCL2, DDX6, or GULP1.

The invention also relates to a method of treating a small cell lung cancer subject, comprising detecting one or more of SLFN11, SIL1, SLC25A3, MAF, AP3B1, C1orf50, BCL2, DDX6, and GULP1, in a tumor cell sample from the subject, and administering an effective amount of a PARP inhibitor to the subject.

In another aspect, the invention relates to a method of selecting a small cell lung cancer subject for PARP inhibitor chemotherapy, comprising detecting one or more of SLFN11, SIL1, SLC25A3, MAF, AP3B1, C1orf50, BCL2, DDX6, or GULP1 in a small cell lung cancer tumor sample of the subject. The method optionally further comprises administering an effective amount of the PARP inhibitor to the subject. In other aspects, the invention relates to a method of selecting a small cell lung cancer subject for PARP inhibitor chemotherapy, comprising detecting SLFN11 expression in the subject, and optionally further comprising administering an effective amount of a PARP inhibitor to the subject. In another aspect, the invention relates to a method of selecting a small cell lung cancer subject for talazoparib chemotherapy, comprising detecting one or more of SLFN11, SIL1, SLC25A3, MAF, AP3B1, C1orf50, BCL2, DDX6, or GULP1 in a SCLC tumor sample from the subject. The method optionally further comprises administering an effective amount of talazoparib or a pharmaceutically acceptable salt thereof to the subject. In other aspects, the invention relates to a method of selecting a small cell lung cancer subject for talazoparib chemotherapy, comprising detecting SLFN11 expression in the subject, and optionally further comprising administering an effective amount of talazoparib or a pharmaceutically acceptable salt thereof to the subject.

In another aspect, the invention relates to a method of treating a human subject having small cell lung cancer with a PARP inhibitor, comprising:

(a) performing a nucleic acid-based detection assay to detect the mRNA expression level of one or more genes selected from the group consisting of SLFN11, SIL1, SLC25A3, MAF, AP3B1, C1orf50, BCL2, DDX6, and GULP1, in cells of a biological sample from the human subject by detecting mRNA expression;
(b) determining that the cells from the human subject express said one or more genes at a level greater than the expression level of the respective genes in cells of a biological sample from a healthy human control; and
(c) administering an effective amount of a PARP inhibitor to the human subject expressing the one or more genes at a level greater than the expression level of the respective genes in cells of a biological sample from a healthy human control, thereby treating small cell lung cancer in said human subject.

In another aspect, the invention relates to a method for diagnosing and treating SCLC in a human subject, the method comprising:

(a) performing a nucleic acid-based detection assay to detect the mRNA expression level of one or more genes selected from the group consisting of SLFN11, SIL1, SLC25A3, MAF, AP3B1, C1orf50, BCL2, DDX6, and GULP1, in cells of a biological sample from the human subject by detecting mRNA expression;
(b) determining that the cells from the human subject express said one or more genes at a level greater than the expression level of the respective genes in cells of a biological sample from a healthy human control; and
(c) administering an effective amount of a PARP inhibitor to the human subject expressing the one or more genes at a level greater than the expression level of the respective genes in cells of a biological sample from a healthy human control, thereby treating small cell lung cancer in the human subject.

In another aspect, the invention relates to a method of diagnosing SCLC in a subject, comprising detecting expression of SLFN11 in the subject. The method optionally further comprises detecting one or more of SIL1, SLC25A3, MAF, AP3B1, C1orf50, BCL2, DDX6, or GULP1 in the subject.

In other aspects, the invention relates to a method of treating a small cell lung cancer subject with a reduced expression level of ATM, comprising administering to the subject an effective amount of a PARP inhibitor or talazoparib or a pharmaceutically acceptable salt thereof. In other aspects, ATM expression in the subject is detected in addition to the one or more detection targets described herein. In other aspects, ATM expression in the subject is reduced.

Additional embodiments, features, and advantages of the invention will be apparent from the following detailed description and through practice of the invention.

For the sake of brevity, the disclosures of the publications cited in this specification, including patents, are herein incorporated by reference.

### BRIEF DESCRIPTION OF THE FIGURES

The present application can be understood by reference to the following description taken in conjunction with the accompanying figures.

FIG. 1 illustrates the correlation of GI₅₀ values between talazoparib and cisplatin sensitivity in various small-cell lung cancer (SCLC) cell lines. GI₅₀ values in log 10 scale for talazoparib appear along the x-axis and for cisplatin along the y-axis. A linear regression line is shown, and the Spearman correlation and p-value are listed as R and P, respectively.

FIG. 2 illustrates the sensitivity of cell lines to talazoparib. Circles without arrows indicate cell lines that were studied for five days; circles with arrows indicate cell lines that were studied for seven days. The size of the data points is set based on the log₁₀(GI₅₀) (lower GI₅₀=smaller size circles).

FIG. 3A illustrates the robust multi-array average (RMA) scores for SLFN11 expression in various small-cell lung cancer lines. Cell lines with an RMA score above 6 (above the line, designated “high” RMA) are differentiated from cell lines with an RMA score below 6 (below the line, designated “low” RMA). FIG. 3B illustrates boxplots of maximum growth inhibition and GI₅₀ for the cell lines treated with talazoparib, pooled by high or low RMA score. The p-values shown are based on an Anova test. FIG. 3C illustrates a Waterfall plot of small-cell lung cancer (SCLC) cell lines ranked by maximum growth inhibition by talazoparib. Bars without arrows correspond to cell lines designated as “high” RMA and bars with arrows correspond to cell lines designated as “low” RMA. FIG. 3D illustrates the correlation between SLFN11 RMA expression score and GI₅₀ for talazoparib for the tested cell lines. A linear regression line is shown, and the Spearman correlation and p-value are listed as R and P, respectively. FIG. 3E illustrates the correlation between SLFN11 RMA expression score and maximum growth inhibition for the tested cell lines. A linear regression line is shown, and the Spearman correlation and p-value are listed as R and P, respectively.

FIG. 4 illustrates top gene expression features associated with talazoparib sensitivity in the 38 NCI SCLC cell lines. Those with nominal p values<0.001 are highlighted in the box in the table. The table columns include: genename=entrez gene symbol; log FC=log fold change of sensitive/resistant cell line groups; t=t statistic; P.value-=nominal p value based on moderated t test; adj.P.val=adjusted pvalue based on FDR. The genes highlighted in the box were plotted by heatmap, which shows a hierarchical clustering using the top nine genes. The bar above the heatmap identifies the cell line sensitivity groups, where “R” is the resistant group and “S” is the sensitive group.

FIG. 5 illustrates a Western blot of SLFN11 protein in 12 small-cell lung cancer (SCLC) cell lines. SLFN11gene expression data from CCLE is listed in the table below the blot to correlate with protein level.

FIGS. 6A, 6B, and 6C illustrate the mean tumor volumes over time for NCI-H1048 (FIG. 6A), NCI-H209 (FIG. 6B), and NCI-H69 (FIG. 6C) small-cell lung cancer (SCLC) xenografts treated with vehicle (triangles), cisplatin (circles, FIGS. 6A and 6B only), and talazoparib (BMN 673; squares).

FIG. 7 illustrates the effect of talazoparib daily dosing on mean tumor volume (measured as change from baseline) for 12 SCLC PDX xenograft models.

FIGS. 8A-8F illustrate the tumor growth curves of individual animals with partial response (FIGS. 8A, 8B), stable disease (FIGS. 8C, 8D) and progressive disease (FIGS. 8E, 8F) after daily dosing with vehicle (circles with solid lines) or BMN 673 (triangles with dotted lines).

FIG. 9A illustrates a regression analysis of single agent talazoparib treatment of 12 PDX xenograft models. The results are grouped as progressive disease (PD, n=6), stable disease (SD, n=3), or partial response (PR, n=3) with talazoparib treatment. Diagonal lines indicate positive values while absence of diagonal lines indicate negative values. The FIG. 9B illustrates the expression of SLFN11protein for 12 PDX models with progressive disease (PD, n=6), stable disease (SD, n=3), or partial response (PR, n=3) with talazoparib. The p-value shown is based on an Anova test. FIG. 9C illustrates the expression of SLFN11 by RNA sequencing analysis (nominalized count (log 2)) across the 12 PDX xenograft models for progressive disease (PD, n=6), stable disease (SD, n=3), or partial response (PR, n=3) with talazoparib treatment. The p-value shown is based on an Anova test.

FIG. 10A illustrates the expression of ATM protein for the PD, SD, and PR groups of PDX xenograft models. FIG. 10B illustrates the expression of ATM in the 12 PDX xenograft models by RNA-seq analysis.

FIG. 11 illustrates the correlation between HRD score and talazoparib sensitivity (GI₅₀). A linear regression line is shown, and the Spearman correlation and p-value are listed as R and P, respectively.

### DETAILED DESCRIPTION OF THE INVENTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

In one aspect, the invention is directed to a method of treating small cell lung cancer in a subject expressing SLFN11, comprising administering to the subject an effective amount of a PARP inhibitor.

In another aspect, the invention is directed to a method of treating small cell lung cancer in a subject expressing SLFN11, comprising administering to the subject an effective amount of talazoparib or a pharmaceutically acceptable salt thereof.

In another aspect, the invention is directed to a method of selecting a small cell lung cancer subject for PARP inhibitor chemotherapy, comprising detecting one or more of SLFN11, SIL1, SLC25A3, MAF, AP3B1, C1orf50, BCL2, DDX6, or GULP1 in a SCLC tumor sample of the subject, and administering an effective amount of a PARP inhibitor to the subject. In some embodiments of the selection method, SLFN11 is detected.

In some embodiments, the subject expresses SLFN11. In other embodiments, the subject expresses one or more of SIL1, SLC25A3, MAF, AP3B1, C1orf50, BCL2, DDX6, or GULP1. In some embodiments, the subject has an increased expression level of one or more of SLFN11, SIL1, SLC25A3, MAF, AP3B1, C1orf50, BCL2, DDX6, or GULP1. In some embodiments, the subject expresses ATM. In other embodiments, the subject exhibits a reduced level of ATM expression. In certain embodiments, the subject expresses the TP53 and/or RB1 mutation. In some embodiments, the detecting step described herein further comprises detecting ATM, or detecting a reduced level of expression of ATM.

In some embodiments, the RMA score for SLFN11 in the subject is 4 or higher, or is 5 or higher, or is 6 or higher, or is 7 or higher, or is 8 or higher. The RMA score is determined based on methods known to one of ordinary skill in the art, in particular, by using the methods described in the examples herein.

In some embodiments, the subject has a Myriad HRD score of 40 or lower, or of 35 or lower, or of 30 or lower, or of 25 or lower, or of 20 or lower. Determining the Myriad HRD score is accomplished using methods known to one of ordinary skill in the art optionally using commercially available test kits.

In some embodiments of the inventive methods, the subject has advanced SCLC. In other embodiments, the subject has been treated previously or is being treated concurrently with a platinum drug such as cisplatin or carboplatin, optionally in combination with etoposide.

In some embodiments, the PARP inhibitor is any compound that inhibits PARP activity. In other embodiments, the PARP inhibitor is talazoparib, olaparib, rucaparib, veliparib, CEP9722, MK4827, or BGB-290, or a pharmaceutically acceptable salt thereof. In other embodiments, the PARP inhibitor is talazoparib or a pharmaceutically acceptable salt thereof. In further embodiments, the PARP inhibitor is the tosylate salt of talazoparib. Talazoparib has the structure shown below:

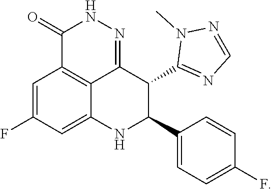 [SMILES: Cn1ncnc1[C@H]1c2n[nH]c(=O)c3cc(F)cc(c23)N[C@@H]1c1ccc(F)cc1]

In some embodiments, talazoparib or a pharmaceutically acceptable salt thereof is administered orally, once daily, at a dose of about 25 to about 1100 μg/day, or about 0.5 to about 2 mg per day, or of about 1 mg/day, or about 0.10 to 0.75 mg/kg/day, or about 0.25-0.30 mg/kg/day. Dosage figures provided herein refer to the dose of the free base form of talazoparib, or are calculated as the free base equivalent of an administered talazoparib salt form. For example, a dosage of 1 mg of talazoparib tosylate refers to talazoparib tosylate in an amount equal to 1 mg free base equivalent of talazoparib.

In some embodiments, the PARP inhibitor is administered in combination with one or more chemotherapeutic agents, surgery, and/or radiation. In other embodiments, the one or more chemotherapeutic agents are selected from the group consisting of a DNA damaging agent, temozolomide, a topoisomerase 1 inhibitor, irinotecan, topotecan, a topoisomerase 2 inhibitor, etoposide, enzalutamide, an ATR inhibitor, an EGFR inhibitor, a platinum drug, cisplatin, carboplatin, and etoposide.

In some embodiments, the one or more biomarkers are detected by an immunohistological assay, an immunohistochemistry staining (IHC) assay, an in-situ LC/MS assay, a promoter methylation assay, a cytological assay, an mRNA expression assay, an RT-PCR assay, a northern blot assay, a protein expression immunosorbent assay (ELISA), an enzyme-linked immunospot assay (ELISPOT), a lateral flow test assay, an enzyme immunoassay, a fluorescent polarization immunoassay, a chemiluminescent immunoassay (CLIA), or a fluorescence activated sorting assay (FACS).

In some embodiments, the expression level of one or more biomarkers in a test sample from a subject is determined using methods known to one of ordinary skill in the art, and the expression level of each biomarker is compared with the expression level of the corresponding biomarker in a normal sample or standard sample. In some embodiments, an increased level of expression of the test sample in relation to that of the normal sample or standard sample indicates that the subject is likely to respond to PARP inhibitor therapy. In some embodiments, an increased level of expression of one or more genes or proteins indicates responsiveness to PARP inhibitor therapy, where the one or more genes or proteins is selected from the group consisting of SLFN11, SIL1, SLC25A3, MAF, AP3B1, C1orf50, BCL2, DDX6, and GULP1. In other embodiments, one gene or protein is SLFN11.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only,” and the like, in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Concentrations that are given as percentages refer to mass ratios, unless indicated differently.

As used herein, a “subject” refers to a human or animal, including all mammals such as primates (particularly higher primates), sheep, dogs, rodents (e.g., mice or rats), guinea pigs, goats, pigs, cats, rabbits, and cows. In some embodiments, the subject is a human. In other embodiments, the subject is a human that may be considered at high-risk for developing SCLC, including an individual who is a current or former smoker. In certain embodiments, the subject is suffering from or has been diagnosed with SCLC. As used herein, “individual” refers to a subject or patient. A healthy or normal individual is an individual in which the disease or condition of interest (including, for example, lung diseases, lung-associated diseases, or other lung conditions) is not detectable by conventional diagnostic methods.

A “biological sample,” “sample,” and “test sample” are used interchangeably herein, and can be any organ, tissue, cell, or cell extract isolated from a subject, such as a sample isolated from a mammal having a lung cancer or at risk for a lung cancer (e.g., based on family history or personal history, such a heavy smoking). For example, a sample can include, without limitation, cells or tissue (e.g., from a biopsy or autopsy) from solid lung tumors, sputum, cough, bronchoalveolar lavage, bronchial brushings, buccal mucosa, peripheral blood, whole blood, red cell concentrates, platelet concentrates, leukocyte concentrates, blood cell proteins, blood plasma, platelet-rich plasma, a plasma concentrate, a precipitate from any fractionation of the plasma, a supernatant from any fractionation of the plasma, blood plasma protein fractions, purified or partially purified blood proteins or other components, serum, tissue or fine needle biopsy samples, and pleural fluid, and the like, isolated from a mammal with a lung cancer, or any other specimen, or any extract thereof, obtained from a patient (human or animal), test subject, healthy volunteer, or experimental animal. Sample sources include blood (including whole blood, leukocytes, peripheral blood mononuclear cells, buffy coat, plasma, and serum), sputum, tears, mucus, nasal washes, nasal aspirate, breath, urine, semen, saliva, peritoneal washings, cystic fluid, meningeal fluid, amniotic fluid, glandular fluid, lymph fluid, cytologic fluid, ascites, pleural fluid, nipple aspirate, bronchial aspirate, bronchial brushing, synovial fluid, joint aspirate, organ secretions, cells, a cellular extract, and cerebrospinal fluid. Samples also include experimentally separated fractions of all of the preceding biological sources. For example, a blood sample can be fractionated into serum or plasma, or into fractions containing particular types of blood cells, such as red blood cells or white blood cells (leukocytes). If desired, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable method for obtaining a biological sample can be employed; exemplary methods include, e.g., phlebotomy, swab (e.g., buccal swab), surgery, biopsy, and a fine needle aspirate biopsy procedure. Exemplary tissues susceptible to fine needle aspiration include lymph node, lung, lung washes, BAL (broncho-alveolar lavage), pleura, thyroid, breast, pancreas, and liver. Samples can also be collected, for example, by micro-dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual. A sample may also include sections of tissues such as frozen sections taken for histological purposes. A “sample” may also be a cell or cell line created under experimental conditions, that is not directly isolated from a subject. A “control” or “reference” includes a sample obtained for use in determining base-line expression or activity. Accordingly, a control sample may be obtained by a number of means including from non-cancerous cells or tissue, e.g., from cells surrounding a tumor or cancerous cells of a subject; from subjects not having a cancer; from subjects not suspected of being at risk for a cancer; or from cells or cell lines derived from such subjects. A control also includes a previously established standard, such as a previously characterized SCLC. Accordingly, any test or assay conducted according to the invention may be compared with the established standard and it may not be necessary to obtain a control sample for comparison each time.

Further, it should be realized that a biological sample can be derived by taking biological samples from a number of individuals and pooling them or pooling an aliquot of each individual's biological sample. The pooled sample can be treated as a sample from a single individual and if the presence of cancer is established in the pooled sample, then each individual biological sample can be re-tested for relevant results.

As used herein, the terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, with disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. Polypeptides can be single chains or associated chains. Also included within the definition are preproteins and intact mature proteins; peptides or polypeptides derived from a mature protein; fragments of a protein; splice variants; recombinant forms of a protein; protein variants with amino acid modifications, deletions, or substitutions; digests; and post-translational modifications, such as glycosylation, acetylation, phosphorylation, and the like.

The invention provides biomarkers, e.g., nucleic acid molecules and expression products thereof, that are differentially expressed in histologically normal cells derived from subjects having a lung cancer and/or in malignant lung cancer cells, compared to normal cells derived from subjects without cancer.

A “biomarker” is a molecular indicator of a specific biological property and as used herein is a nucleic acid molecule (e.g., a gene or gene fragment) or an expression product thereof (e.g., a polypeptide or peptide fragment or variant thereof) whose differential expression (presence, absence, over-expression, or under-expression relative to a reference) within a cell or tissue indicates the presence or absence of a small cell lung cancer, or the increased or decreased sensitivity to PARP inhibitor exposure. An “expression product” as used herein is a transcribed sense or antisense RNA molecule (e.g., an mRNA), or a translated polypeptide corresponding to or derived from a polynucleotide sequence. In some embodiments, an expression product can refer to an amplification product (amplicon) or cDNA corresponding to the RNA expression product transcribed from the polynucleotide sequence. Biomarkers are detectable and measurable by a variety of methods including laboratory assays and medical imaging. When a biomarker is a protein, it is also possible to use the expression of the corresponding gene as a surrogate measure of the amount or presence or absence of the corresponding protein biomarker in a biological sample or methylation state of the gene encoding the biomarker or proteins that control expression of the biomarker.

By “differential expression” or “differentially expressed” is meant a difference in the frequency or quantity, or both, of a biomarker in a cell or tissue or sample derived from a subject having a lung cancer compared to a reference cell or tissue or sample, e.g., in a malignant lung cancer cell and/or in a normal cell derived from a subject having a lung cancer (i.e., a cell having a malignancy associated change) compared to a reference or normal cell, e.g., a cell derived from a subject without cancer or with undetectable cancer or a normal cell derived from a subject who has undergone successful resection of lung cancer. In some embodiments, the control or reference cell may be a SCLC or a NSCLC. In some embodiments, differential expression refers to a difference in the frequency or quantity, or both, of a biomarker in a malignant lung cancer cell compared to the reference cell. For example, differential expression of a biomarker can refer to an elevated level or a decreased level of expression of the biomarker in samples of lung cancer patients compared to samples of reference subjects, e.g., measurement of protein level or antibody titer in blood, urine, saliva, serum, pleural effusions or bronchoalveolar lavages samples taken from lung cancer patients compared to the measurement of protein level or antibody titer in blood, urine, saliva, serum, pleural effusions, or bronchoalveolar lavage samples taken from non-lung cancer controls, including healthy subjects and subjects with respiratory airway infections like bronchitis and bronchiolitis. Alternatively or additionally, differential expression of a biomarker can refer to detection at a higher frequency or at a lower frequency of the biomarker in samples of lung cancer patients compared to samples of reference subjects. A biomarker can be differentially present in terms of quantity, frequency, or both. In some embodiments, differential expression of the biomarkers of the invention may be measured at different time points, e.g., before and after therapy. By “level of expression” or “expressing level” is meant the level of mRNA, as well as pre-mRNA nascent transcript(s), transcript processing intermediates, mature mRNA(s), and degradation products, encoded by a gene in the cell, and/or the level of protein, protein fragments, and degradation products in a cell. Suitable comparisons may also be made to a level of the detection product in a normal cell in the same patient, or by comparison to an historical database.

The difference in quantity or frequency or both of a biomarker may be measured by any suitable technique, such as a statistical technique. For example, a biomarker can be differentially expressed between a lung cancer sample and a reference sample, if the frequency of detecting the biomarker in a lung cancer sample is significantly higher or lower than in the reference sample, as measured by standard statistical analyses such as student's t-test or an Anova test, where p<0.05 is generally considered statistically significant. In some embodiments, a biomarker is differentially expressed if it is detected at a level more or less frequently in a small cell lung cancer compared to a reference sample; for example, detection may be at least about 1, 5, 10, 20, 30, 40, 50, 60, 70, 80, 90, 100% or more, or 2-, 5-, 10-, or more fold, more or less frequently in a lung cancer compared to a reference sample. Alternatively or additionally, a biomarker is differentially expressed if the amount of the biomarker in a lung cancer is statistically significantly different, e.g., by more or less than the amount of the biomarker in the reference sample, for example, at least 1, 5, 10, 20, 30, 40, 50, 60, 70, 80, 90, 100% or more, or 2-, 5-, 10-, or more fold, when compared to the amount of the biomarker in a reference sample or if it is detectable in one sample and not detectable in the other. In some embodiments, differential expression may refer to an increase or decrease in expression, which may be an increase or decrease of at least 1, 5, 10, 20, 30, 40, 50, 60, 70, 80, 90, 100% or more, or 2-, 5-, 10- or more fold, in a test sample relative to a reference sample.

Biomarkers for identifying small cell lung cancer subjects sensitive to PARP inhibitors, according to the invention, include SLFN11, SIL1, SLC25A3, MAF, AP3B1, C1orf50, BCL2, DDX6, and GULP1. Two or more of these biomarkers, e.g., 2, 3, 4, 5, 6, 7, 8, or 9 of the biomarkers, may be used together in any combination in an assay according to the invention. In some embodiments, one or more of the biomarkers may be specifically excluded from an assay. In some embodiments, particular combinations will be used, for example in differentiating SCLC and NSCLC or determining sensitivity to a PARP inhibitor or talazoparib. In a particular embodiment of the present invention SLFN11 is used, or SLFN11 is used in combination with at least one or more of the biomarkers selected from the group consisting of SIL1, SLC25A3, MAF, AP3B1, C1orf50, BCL2, DDX6, and GULP1.

Biomarkers according to the invention include substantially identical homologues and variants of the nucleic acid molecules and expression products thereof described herein, for example, a molecule that includes nucleotide sequences encoding polypeptides functionally equivalent to the biomarkers of the invention, e.g., sequences having one or more nucleotide substitutions, additions, or deletions, such as allelic variants or splice variants or species variants or molecules differing from the nucleic acid molecules and polypeptides referred to herein due to the degeneracy of the genetic code. Species variants are nucleic acid sequences that vary from one species to another, although the resulting polypeptides generally will have significant amino acid identity and functional similarity relative to each other. A polymorphic variant (e.g., a single nucleotide polymorphism or SNP) is a variation in the nucleic acid sequence of a particular gene between individuals of a given species.

A “substantially identical” sequence is an amino acid or nucleotide sequence that differs from a reference sequence only by one or more conservative substitutions, as discussed herein, or by one or more non-conservative substitutions, deletions, or insertions located at positions of the sequence that do not destroy the biological function of the amino acid or nucleic acid molecule. Such a sequence can be any integer from 10% to 99%, or more generally at least 10%, 20%, 30%, 40%, 50, 55%, or 60%, or at least 65%, 75%, 80%, 85%, 90%, or 95%, or as much as 96%, 97%, 98%, or 99% identical when optimally aligned at the amino acid or nucleotide level to the sequence used for comparison using, for example, the Align Program (Myers and Miller, CABIOS, 1989, 4:11-17) or FASTA. For polypeptides, the length of comparison sequences may be at least 2, 5, 10, or 15 amino acids, or at least 20, 25, or 30 amino acids. In alternate embodiments, the length of comparison sequences may be at least 35, 40, or 50 amino acids, or over 60, 80, or 100 amino acids, or for the entire length of the protein. For nucleic acid molecules, the length of comparison sequences may be at least 5, 10, 15, 20, or 25 nucleotides, or at least 30, 40, or 50 nucleotides. In alternate embodiments, the length of comparison sequences may be at least 60, 70, 80, or 90 nucleotides, or over 100, 200, or 500 nucleotides. Sequence identity can be readily measured using publicly available sequence analysis software (e.g., Sequence Analysis Software Package of the Genetics Computer Group, University of Wisconsin Biotechnology Center, 1710 University Avenue, Madison, Wis. 53705, or BLAST software available from the National Library of Medicine, or as described herein). Examples of useful software include the programs Pile-up and PrettyBox. Such software matches similar sequences by assigning degrees of homology to various, deletions, substitutions, and other modifications. Alternatively, or additionally, two nucleic acid sequences may be “substantially identical” if they hybridize under high stringency conditions. In some embodiments, high stringency conditions are, for example, conditions that allow hybridization comparable with the hybridization that occurs using a DNA probe of at least 500 nucleotides in length, in a buffer containing 0.5 M NaHPO4, pH 7.2, 7% SDS, 1 mM EDTA, and 1% BSA (fraction V), at a temperature of 65° C., or a buffer containing 48% formamide, 4.8×SSC, 0.2 M Tris-Cl, pH 7.6, Ix Denhardt's solution, 10% dextran sulfate, and 0.1% SDS, at a temperature of 42° C. (These are typical conditions for high stringency northern or Southern hybridizations.) Hybridizations may be carried out over a period of about 20 to 30 minutes, or about 2 to 6 hours, or about 10 to 15 hours, or over 24 hours or more. High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al, Current Protocols in Molecular Biology, John Wiley & Sons, New York, N.Y., 1998, which is hereby incorporated by reference.

### Preparation of Reagents Using Biomarkers

The biomarkers described herein may be used to prepare oligonucleotide probes and antibodies that hybridize to or specifically bind the biomarkers described herein, and homologues and variants thereof.

Antibodies

An “antibody” includes molecules having antigen-binding regions, such as whole antibodies of any isotype (IgG, IgA, IgM, IgE, etc.), polyclonal antibodies, and fragments thereof. Antibody fragments include Fab′, Fab, F(ab′)2, single domain antibodies, Fv, scFv, and the like. Antibodies may be prepared using standard techniques of preparation as, for example, described in Harlow and Lane (Harlow and Lane Antibodies; A Laboratory Manual, Cold Spring Harbor Laboratory, Cold Spring Harbor, N.Y., 1988), or known to those skilled in the art. For example, a coding sequence for a polypeptide biomarker of the invention may be purified to the degree necessary for immunization of rabbits. To attempt to minimize the potential problems of low affinity or specificity of antisera, two or three polypeptide constructs may be generated for each protein, and each construct may be injected into at least two rabbits. Antisera may be raised by injections in a series, preferably including at least three booster injections. Primary immunizations may be carried out with Freund's complete adjuvant and subsequent immunizations with Freund's incomplete adjuvant. Antibody titers may be monitored by Western blot and immunoprecipitation analyses using the purified protein. Immune sera may be affinity purified using CNBr-Sepharose-coupled protein. Antiserum specificity may be determined using a panel of unrelated proteins. Antibody fragments may be prepared recombinantly or by proteolytic cleavage. Peptides corresponding to relatively unique immunogenic regions of a polypeptide biomarker of the invention may be generated and coupled to keyhole limpet hemocyanin (KLH) through an introduced C-terminal lysine. Antiserum to each of these peptides may be affinity purified on peptides conjugated to BSA, and specificity tested in ELISA and Western blots using peptide conjugates and by Western blot and immunoprecipitation.

Monoclonal antibodies, which specifically bind any one of the polypeptide biomarkers of the invention are prepared according to Standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., In Monoclonal Antibodies and T Cell Hybridomas, Elsevier, N.Y., 1981). Alternatively monoclonal antibodies may be prepared using the polypeptides of the invention and a phage display library (Vaughan et al., Nature Biotech 14:309-314, 1996). Once produced, monoclonal antibodies may also be tested for specific recognition by Western blot or immunoprecipitation.

In some embodiments, antibodies may be produced using polypeptide fragments that appear likely to be immunogenic, by criteria such as high frequency of charged residues. Antibodies can be tailored to minimize adverse host immune response by, for example, using chimeric antibodies that contain an antigen binding domain from one species and the Fc portion from another species, or by using antibodies made from hybridomas of the appropriate species. For example, with SLFN11, the antibodies are tailored to be specific for a certain SLFN11 region.

An antibody “specifically binds” an antigen when it recognizes and binds the antigen, for example, a biomarker as described herein, but does not substantially recognize and bind other molecules in a sample. Such an antibody has, for example, an affinity for the antigen, which is at least 2, 5, 10, 100, 1000, or 10000 times greater than the affinity of the antibody for another reference molecule in a sample. Specific binding to an antibody under such conditions may require an antibody that is selected for its specificity for a particular biomarker. For example, a polyclonal antibody raised to a biomarker from a specific species such as rat, mouse, or human may be selected for only those polyclonal antibodies that are specifically immunoreactive with the biomarker and not with other proteins, except for polymorphic variants and alleles of the biomarker. In some embodiments, a polyclonal antibody raised to a biomarker from a specific species such as rat, mouse, or human may be selected for only those polyclonal antibodies that are specifically immunoreactive with the biomarker from that species and not with other proteins, including polymorphic variants and alleles of the biomarker. Antibodies that specifically bind any of the biomarkers described herein may be employed in an immunoassay by contacting a sample with the antibody and detecting the presence of a complex of the antibody bound to the biomarker in the sample. The antibodies used in an immunoassay may be produced as described herein or known in the art, or may be commercially available from suppliers, such as Dako Canada, Inc., Mississauga, ON. The antibody may be fixed to a solid substrate (e.g., nylon, glass, ceramic, plastic, and the like) before being contacted with the sample, to facilitate subsequent assay procedures. The antibody-biomarker complex may be visualized or detected using a variety of standard procedures, such as detection of radioactivity, fluorescence, luminescence, chemiluminescence, absorbance, or by microscopy, imaging, and the like. Immunoassays include immunohistochemistry, enzyme-linked immunosorbent assay (ELISA), western blotting, immunoradiometric assay (IRMA), lateral flow, evanescence (DiaMed AG, Cressier surMorat, Switzerland, as described in European Patent Publications EP1371967, EP1079226 and EP1204856), immunohisto/cyto-chemistry, and other methods known to those of skill in the art. Immunoassays can be used to determine presence or absence of a biomarker in a sample as well as the amount of a biomarker in a sample. The amount of an antibody-biomarker complex can be determined by comparison to a reference or standard, such as a polypeptide known to be present in the sample. The amount of an antibody-biomarker complex can also be determined by comparison to a reference or standard, such as the amount of the biomarker in a reference or control sample. Accordingly, the amount of a biomarker in a sample need not be quantified in absolute terms, but may be measured in relative terms with respect to a reference or control.

Probes and Primers

A “probe” or “primer” is a single-stranded DNA or RNA molecule of defined sequence that can base pair to a second DNA or RNA molecule that contains a complementary sequence (the target). The stability of the resulting hybrid molecule depends upon the extent of the base pairing that occurs, and is affected by parameters such as the degree of complementarity between the probe and target molecule, and the degree of stringency of the hybridization conditions. The degree of hybridization stringency is affected by parameters such as the temperature, salt concentration, and concentration of organic molecules, such as formamide, and is determined by methods that are known to those skilled in the art. Probes or primers specific for the nucleic acid biomarkers described herein, or portions thereof, may vary in length by any integer from at least 8 nucleotides to over 500 nucleotides, including any value in between, depending on the purpose for which, and conditions under which, the probe or primer is used. For example, a probe or primer may be 8, 10, 15, 20, or 25 nucleotides in length, or may be at least 30, 40, 50, or 60 nucleotides in length, or may be over 100, 200, 500, or 1000 nucleotides in length. Probes or primers specific for the nucleic acid biomarkers described herein may have greater than 20-30% sequence identity, or at least 55-75% sequence identity, or at least 75-85% sequence identity, or at least 85-99% sequence identity, or 100% sequence identity to the nucleic acid biomarkers described herein. Probes or primers may be derived from genomic DNA or cDNA, for example, by amplification, or from cloned DNA segments, and may contain either genomic DNA or cDNA sequences representing all or a portion of a single gene from a single individual. A probe may have a unique sequence (e.g., 100% identity to a nucleic acid biomarker) and/or have a known sequence. Probes or primers may be chemically synthesized. A probe or primer may hybridize to a nucleic acid biomarker under high stringency conditions as described herein.

Probes or primers can be detectably-labeled, either radioactively or non-radioactively, by methods that are known to those skilled in the art. Probes or primers can be used for lung cancer detection methods involving nucleic acid hybridization, such as nucleic acid sequencing, nucleic acid amplification by the polymerase chain reaction (e.g., RT-PCR), single stranded conformational polymorphism (SSCP) analysis, restriction fragment polymorphism (RFLP) analysis, Southern hybridization, northern hybridization, in situ hybridization, electrophoretic mobility shift assay (EMSA), fluorescent in situ hybridization (FISH), and other methods that are known to those skilled in the art.

By “detectably labeled” is meant any means for marking and identifying the presence of a molecule, e.g., an oligonucleotide probe or primer, a gene or fragment thereof, or a cDNA molecule. Methods for detectably-labeling a molecule are well known in the art and include, without limitation, radioactive labeling (e.g., with an isotope such as 32P or 35S) and nonradioactive labeling such as, enzymatic labeling (for example, using horseradish peroxidase or alkaline phosphatase), chemiluminescent labeling, fluorescent labeling (for example, using fluorescein), bioluminescent labeling, or antibody detection of a ligand attached to the probe. Also included in this definition is a molecule that is detectably labeled by an indirect means, for example, a molecule that is bound with a first moiety (such as biotin) that is, in turn, bound to a second moiety that may be observed or assayed (such as fluorescein-labeled streptavidin). Labels also include digoxigenin, luciferases, and aequorin.

Arrays and Kits

Antibodies, probes, primers and other reagents prepared using the biomarkers of the invention may be used to prepare arrays for use in detecting lung cancer. By “array” or “matrix” is meant refer to a pattern or arrangement of addressable locations or “addresses,” each representing an independent site, on a surface. Arrays generally require a solid support (for example, nylon, glass, ceramic, plastic, and the like) to which the nucleic acid molecules, polypeptides, antibodies, tissue, and the like, are attached in a specified dimensional arrangement, such that the pattern of hybridization to a probe is easily determinable.

Generally, a probe (e.g., an antibody, nucleic acid probe or primer, polypeptide, and the like) is immobilized on an array surface and contacted with a sample containing a target binding partner (e.g., in the case of an antibody, a polypeptide that specifically binds the antibody, or in the case of a probe, a nucleic acid molecule that hybridizes to the probe) under conditions suitable for binding. If desired, unbound material in the sample may be removed. The bound target is detected and the binding results are analyzed using appropriate statistical or other methods. The probe or the target may be detectably labeled for ease of detection and subsequent analysis. Multiple probes corresponding to the biomarkers described herein may be used. The multiple probes may correspond to one or more of the biomarkers described herein. In addition to probes capable of binding the biomarkers described herein, the arrays may control and reference nucleic acid molecules, polypeptides, or antibodies, to allow for normalization of results from one experiment to another and the comparison of multiple experiments on a quantitative level. Accordingly, the invention provides biological assays using nucleic acid, polypeptide, antibody, or cytology arrays.

The invention also provides kits for detecting small cell lung cancer and particularly with respect to the gene expression motifs identified herein. The kits may include one or more reagents corresponding to the biomarkers described herein, e.g., antibodies that specifically bind the biomarkers secreted as antigens in the body fluids, recombinant proteins that bind biomarker specific antibodies, nucleic acid probes or primers that hybridize to the biomarkers. In some embodiments, the kits may include a plurality of reagents, e.g., on an array, corresponding to the biomarkers described herein. The kits may include detection reagents, e.g., reagents that are detectably labeled. The kits may include written instructions for use of the kit in (early) detection and subtyping of lung cancer, and may include other reagents and information such as control or reference standards, wash solutions, analysis software, and the like.

### Diagnostic and Other Methods

Small cell lung cancer subjects expressing one or more of the biomarkers identified herein may be diagnosed by detecting the differential expression of one or more of the biomarkers, by immunoassay, such as immunohistochemistry, ELISA, western blotting, or any other method known to those of skill in the diagnostic arts. The detecting may be carried out in vitro or in vivo.

Individual biomarkers and combinations of more than one biomarker are useful diagnostics. In particular, the combination of one or more biomarkers described herein enables accurate (early) diagnosis and subtypes of lung cancer. Variation in differential expression across multiple biomarkers in different samples can diagnose or predict the presence or absence of a particular type of lung cancer, the response to a particular therapy for lung cancer, or better assess the risk for developing a lung cancer. For example, the expression of SLFN11 can be used to detect the presence of SCLC in a sample or to select a patient for PARP inhibitor therapy or talazoparib therapy. Suitable statistical methods and algorithms, e.g., logistical regression algorithm, may be used to analyze and use multiple biomarkers for diagnostic, prognostic, theranostic, or other purposes. The biomarkers (or specific combination of any one or more of the biomarkers) can be detected and measured multiple times, for example, before, during and after a therapy for small cell lung cancer.

Detection of the biomarkers described herein may be performed as an initial screen for the (early) detection and subtyping of lung cancer and/or may be used in conjunction with conventional methods of lung cancer diagnosis, such as sputum cytology, chest X-ray, CT scans, spiral CT, PET, PET-CT with specific tracers e.g. 89Zr, 11C, fluorescent dyes, scintigraphy, biopsy, traditional morphological MACs analysis, and the like. Detection of the biomarkers described herein may also be performed in conjunction with previously recognized biomarkers for lung cancer, such as pRb2/p130, p53, and/or ras. Detection of the biomarkers described herein may be performed as part of a routine examination, for example, of heavy smokers over a certain age (e.g., over 60), or may be performed to determine baseline levels of the biomarkers in subjects at risk for lung cancer (e.g., heavy smokers).

In general, the biomarker panel of the present invention is to be used for molecular imaging (including the aforementioned in vivo imaging techniques) for molecular diagnosis and/or detection and/or to monitor treatment for lung cancer and/or to identify subjects for PARP inhibitor treatment. Detection of the biomarkers described herein may enable a medical practitioner to determine the appropriate course of action for a subject (e.g., further testing, surgery, no action, etc.) based on the diagnosis. Detection of the biomarkers described herein may also help determine the presence or absence of small cell lung cancer, early diagnosis of small cell lung cancer, prognosis for small cell lung cancer, subtyping of small cell lung cancer, evaluation of the efficacy of a therapy for small cell lung cancer, monitoring a small cell lung cancer therapy in a subject, or detecting relapse of small cell lung cancer in a subject who has undergone therapy for small cell lung cancer and is in remission. In alternative aspects, the biomarkers and reagents prepared using the biomarkers may be used to identify SCLC therapeutics. The kits and arrays can be used to measure biomarkers according to the invention, to diagnose and sub type a lung cancer. The kits can also be used to monitor a subject's response to a SCLC therapy, enabling the medical practitioner to modify the treatment based upon the results of the test. The kits can also be used to identify and validate lung cancer therapeutics, such as small molecules, peptides, and the like.

As used herein, “biomarker value,” “value,” “biomarker level,” and “level” are used interchangeably to refer to a measurement that is made using any analytical method for detecting the biomarker in a biological sample and that indicates the presence, absence, absolute amount or concentration, relative amount or concentration, titer, a level, an expression level, a ratio of measured levels, or the like, of, for, or corresponding to the biomarker in the biological sample. The exact nature of the “value” or “level” depends on the specific design and components of the particular analytical method employed to detect the biomarker.

When a biomarker indicates or is a sign of an abnormal process or a disease or other condition in an individual, that biomarker is generally described as being either over-expressed or under-expressed as compared to an expression level or value of the biomarker that indicates or is a sign of a normal process or an absence of a disease or other condition in an individual. “Up-regulation,” “up-regulated,” “over-expression,” “over-expressed,” and any variations thereof, are used interchangeably to refer to a value or level of a biomarker in a biological sample that is greater than a value or level (or range of values or levels) of the biomarker that is typically detected in similar biological samples from healthy or normal individuals. The terms may also refer to a value or level of a biomarker in a biological sample that is greater than a value or level (or range of values or levels) of the biomarker that may be detected at a different stage of a particular disease.

“Down-regulation,” “down-regulated,” “under-expression,” “under-expressed,” and any variations thereof are used interchangeably to refer to a value or level of a biomarker in a biological sample that is less than a value or level (or range of values or levels) of the biomarker that is typically detected in similar biological samples from healthy or normal individuals. The terms may also refer to a value or level of a biomarker in a biological sample that is less than a value or level (or range of values or levels) of the biomarker that may be detected at a different stage of a particular disease.

Further, a biomarker that is either over-expressed or under-expressed can also be referred to as being “differentially expressed” or as having a “differential level” or “differential value” as compared to a “normal” expression level or value of the biomarker that indicates or is a sign of a normal process or an absence of a disease or other condition in an individual. Thus, “differential expression” of a biomarker can also be referred to as a variation from a “normal” expression level of the biomarker.

The term “differential gene expression” and “differential expression” are used interchangeably to refer to a gene (or its corresponding protein expression product) whose expression is activated to a higher or lower level in a subject suffering from a specific disease, relative to its expression in a normal or control subject. The terms also include genes (or the corresponding protein expression products) whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a variety of changes including mRNA levels, surface expression, secretion or other partitioning of a polypeptide. Differential gene expression may include a comparison of expression between two or more genes or their gene products; or a comparison of the ratios of the expression between two or more genes or their gene products; or even a comparison of two differently processed products of the same gene, which differ between normal subjects and subjects suffering from a disease; or between various stages of the same disease. Differential expression includes both quantitative, as well as qualitative, differences in the temporal or cellular expression pattern in a gene or its expression products among, for example, normal and diseased cells, or among cells which have undergone different disease events or disease stages.

As used herein, “detecting” or “determining” with respect to a biomarker value includes the use of both the instrument required to observe and record a signal corresponding to a biomarker value and the material/s required to generate that signal. In various embodiments, the biomarker value is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

“Diagnose,” “diagnosing,” “diagnosis,” and variations thereof, refer to the detection, determination, or recognition of a health status or condition of an individual on the basis of one or more signs, symptoms, data, or other information pertaining to that individual. The health status of an individual can be diagnosed as healthy/normal (e.g., a diagnosis of the absence of a disease or condition) or diagnosed as ill/abnormal (e.g., a diagnosis of the presence, or an assessment of the characteristics, of a disease or condition). The terms “diagnose,” “diagnosing,” “diagnosis,” and the like, encompass, with respect to a particular disease or condition, the initial detection of the disease; the characterization or classification of the disease; the detection of the progression, remission, or recurrence of the disease; and the detection of disease response after the administration of a treatment or therapy to the individual. The diagnosis of SCLC includes distinguishing individuals who have cancer from individuals who do not.

“Prognose,” “prognosing,” “prognosis,” and variations thereof refer to the prediction of a future course of a disease or condition in an individual who has the disease or condition (e.g., predicting patient survival), and such terms encompass the evaluation of disease response after the administration of a treatment or therapy to the individual.

### Exemplary Uses of Biomarkers

In various exemplary embodiments, methods are provided for diagnosing SCLC in an individual by detecting one or more biomarker values corresponding to one or more biomarkers that are present in the circulation of an individual, such as in serum or plasma, by any number of analytical methods, including any of the analytical methods described herein. These biomarkers are, for example, differentially expressed in individuals with SCLC as compared to individuals without SCLC, or are differentially expressed in SCLC subjects who are more likely to be sensitive to PARP inhibitor treatment. Detection of the differential expression of a biomarker in an individual can be used, for example, to permit the early diagnosis of SCLC, or to monitor SCLC recurrence, or for prescription of PARP inhibitor therapy, or for other clinical indications.

Any of the biomarkers described herein may be used in a variety of clinical indications for SCLC, including any of the following: detection of SCLC (such as in a high-risk individual or population); characterizing SCLC (e.g., determining SCLC type, sub-type, or stage), such as by distinguishing between non-small cell lung cancer (NSCLC) and small cell lung cancer (SCLC); determining SCLC prognosis; monitoring SCLC progression or remission; monitoring for SCLC recurrence; monitoring metastasis; treatment selection, in particular for treatment with a PARP inhibitor or talazoparib; monitoring response to a therapeutic agent or other treatment; stratification of individuals for computed tomography (CT) screening (e.g., identifying those individuals at greater risk of SCLC and thereby most likely to benefit from spiral-CT screening, thus increasing the positive predictive value of CT); combining biomarker testing with additional biomedical information, such as smoking history, etc., or with nodule size, morphology, etc. (such as to provide an assay with increased diagnostic performance compared to CT testing or biomarker testing alone); facilitating the diagnosis of a pulmonary nodule as malignant or benign; facilitating clinical decision making once a pulmonary nodule is observed on CT (e.g., ordering repeat CT scans if the nodule is deemed to be low risk, such as if a biomarker-based test is negative, with or without categorization of nodule size, or considering biopsy if the nodule is deemed medium to high risk, such as if a biomarker-based test is positive, with or without categorization of nodule size); and facilitating decisions regarding clinical follow-up (e.g., whether to implement repeat CT scans, fine needle biopsy, nodule resection or thoracotomy after observing a non-calcified nodule on CT). Biomarker testing may improve positive predictive value (PPV) over CT or chest X-ray screening of high risk individuals alone. In addition to their utilities in conjunction with CT screening, the biomarkers described herein can also be used in conjunction with any other imaging modalities used for SCLC, such as chest X-ray, bronchoscopy or fluorescent bronchoscopy, MRI or PET scan. Furthermore, the described biomarkers may also be useful in permitting certain of these uses before indications of SCLC are detected by imaging modalities or other clinical correlates, or before symptoms appear. It further includes distinguishing individuals with indeterminate pulmonary nodules identified with a CT scan or other imaging method, screening of high risk smokers for SCLC, and diagnosing an individual with SCLC.

As an example of the manner in which any of the biomarkers described herein can be used to diagnose SCLC, differential expression of one or more of the described biomarkers in an individual who is not known to have SCLC may indicate that the individual has SCLC, thereby enabling detection of SCLC at an early stage of the disease when treatment is most effective, perhaps before the SCLC is detected by other means or before symptoms appear. Over-expression of one or more of the biomarkers during the course of SCLC may be indicative of SCLC progression, e.g., a SCLC tumor is growing and/or metastasizing (and thus indicate a poor prognosis), whereas a decrease in the degree to which one or more of the biomarkers is differentially expressed (e.g., in subsequent biomarker tests, the expression level in the individual is moving toward or approaching a “normal” expression level) may be indicative of SCLC remission, e.g., a SCLC tumor is shrinking (and thus indicate a good or better prognosis). Similarly, an increase in the degree to which one or more of the biomarkers is differentially expressed (e.g., in subsequent biomarker tests, the expression level in the individual is moving further away from a “normal” expression level) during the course of SCLC treatment may indicate that the SCLC is progressing and therefore indicate that the treatment is ineffective, whereas a decrease in differential expression of one or more of the biomarkers during the course of SCLC treatment may be indicative of SCLC remission and therefore indicate that the treatment is working successfully. Additionally, an increase or decrease in the differential expression of one or more of the biomarkers after an individual has apparently been cured of SCLC may be indicative of SCLC recurrence. In a situation such as this, for example, the individual can be re-started on therapy (or the therapeutic regimen modified such as to increase dosage amount and/or frequency, if the individual has maintained therapy) at an earlier stage than if the recurrence of SCLC was not detected until later. Furthermore, a differential expression level of one or more of the biomarkers in an individual may be predictive of the individual's response to a particular therapeutic agent. In monitoring for SCLC recurrence or progression, changes in the biomarker expression levels may indicate the need for repeat imaging (e.g., repeat CT scanning), such as to determine SCLC activity or to determine the need for changes in treatment.

Detection of any of the biomarkers described herein may be useful following, or in conjunction with, SCLC treatment, such as to evaluate the success of the treatment or to monitor SCLC remission, recurrence, and/or progression (including metastasis) following treatment. SCLC treatment may include, for example, administration of a therapeutic agent to the individual, performance of surgery (e.g., surgical resection of at least a portion of a SCLC tumor or removal of SCLC and surrounding tissue), administration of radiation therapy, or any other type of SCLC treatment used in the art, and any combination of these treatments. Lung cancer treatment may include, for example, administration of a therapeutic agent to the individual, performance of surgery (e.g., surgical resection of at least a portion of a lung tumor), administration of radiation therapy, or any other type of SCLC treatment used in the art, and any combination of these treatments. For example, siRNA molecules are synthetic double stranded RNA molecules that inhibit gene expression and may serve as targeted lung cancer therapeutics. For example, any of the biomarkers may be detected at least once after treatment or may be detected multiple times after treatment (such as at periodic intervals), or may be detected both before and after treatment. Differential expression levels of any of the biomarkers in an individual over time may be indicative of SCLC progression, remission, or recurrence, examples of which include any of the following: an increase or decrease in the expression level of the biomarkers after treatment compared with the expression level of the biomarker before treatment; an increase or decrease in the expression level of the biomarker at a later time point after treatment compared with the expression level of the biomarker at an earlier time point after treatment; and a differential expression level of the biomarker at a single time point after treatment compared with normal levels of the biomarker.

As a specific example, the biomarker levels for any of the biomarkers described herein can be determined in pre-surgery and post-surgery (e.g., 2-16 weeks after surgery) serum or plasma samples. An increase in the biomarker expression level(s) in the post-surgery sample compared with the pre-surgery sample can indicate progression of SCLC (e.g., unsuccessful surgery), whereas a decrease in the biomarker expression level(s) in the post-surgery sample compared with the pre-surgery sample can indicate regression of SCLC (e.g., the surgery successfully removed the lung tumor). Similar analyses of the biomarker levels can be carried out before and after other forms of treatment, such as before and after radiation therapy or administration of a therapeutic agent or cancer vaccine.

In addition to testing biomarker levels as a stand-alone diagnostic test, biomarker levels can also be done in conjunction with determination of SNPs or other genetic lesions or variability that are indicative of increased risk of susceptibility of disease. (See, e.g., Amos et al., Nature Genetics 40, 616-622 (2009)).

In addition to testing biomarker levels as a stand-alone diagnostic test, biomarker levels can also be done in conjunction with radiologic screening, such as CT screening. For example, the biomarkers may facilitate the medical and economic justification for implementing CT screening, such as for screening large asymptomatic populations at risk for SCLC (e.g., smokers). For example, a “pre-CT” test of biomarker levels could be used to stratify high-risk individuals for CT screening, such as for identifying those who are at highest risk for SCLC based on their biomarker levels and who should be prioritized for CT screening. If a CT test is implemented, biomarker levels (e.g., as determined by an aptamer assay of serum or plasma samples) of one or more biomarkers can be measured and the diagnostic score could be evaluated in conjunction with additional biomedical information (e.g., tumor parameters determined by CT testing) to enhance positive predictive value (PPV) over CT or biomarker testing alone. A “post-CT” aptamer panel for determining biomarker levels can be used to determine the likelihood that a pulmonary nodule observed by CT (or other imaging modality) is malignant or benign.

Detection of any of the biomarkers described herein may be useful for post-CT testing. For example, biomarker testing may eliminate or reduce a significant number of false positive tests over CT alone. Further, biomarker testing may facilitate treatment of patients. By way of example, if a lung nodule is less than 5 mm in size, results of biomarker testing may advance patients from “watch and wait” to biopsy at an earlier time; if a lung nodule is 5-9 mm, biomarker testing may eliminate the use of a biopsy or thoracotomy on false positive scans; and if a lung nodule is larger than 10 mm, biomarker testing may eliminate surgery for a sub-population of these patients with benign nodules. Eliminating the need for biopsy in some patients based on biomarker testing would be beneficial because there is significant morbidity associated with nodule biopsy and difficulty in obtaining nodule tissue depending on the location of nodule. Similarly, eliminating the need for surgery in some patients, such as those whose nodules are actually benign, would avoid unnecessary risks and costs associated with surgery.

In addition to testing biomarker levels in conjunction with radiologic screening in high risk individuals (e.g., assessing biomarker levels in conjunction with size or other characteristics of a lung nodule or mass observed on an imaging scan), information regarding the biomarkers can also be evaluated in conjunction with other types of data, particularly data that indicates an individual's risk for SCLC (e.g., patient clinical history, occupational exposure history, symptoms, family history of cancer, risk factors such as whether or not the individual was a smoker, and/or status of other biomarkers, etc.). These various data can be assessed by automated methods, such as a computer program/software, which can be embodied in a computer or other apparatus/device.

Any of the described biomarkers may also be used in imaging tests. For example, an imaging agent can be coupled to any of the described biomarkers, which can be used to aid in SCLC diagnosis, to monitor disease progression/remission or metastasis, to monitor for disease recurrence, or to monitor response to therapy, among other uses.

### Detection and Determination of Biomarkers and Biomarker Values

A biomarker value for the biomarkers described herein can be detected using any of a variety of known analytical methods. In one embodiment, a biomarker value is detected using a capture reagent. As used herein, a “capture agent” or “capture reagent” refers to a molecule that is capable of binding specifically to a biomarker. In various embodiments, the capture reagent can be exposed to the biomarker in solution or can be exposed to the biomarker while the capture reagent is immobilized on a solid support. In other embodiments, the capture reagent contains a feature that is reactive with a secondary feature on a solid support. In these embodiments, the capture reagent can be exposed to the biomarker in solution, and then the feature on the capture reagent can be used in conjunction with the secondary feature on the solid support to immobilize the biomarker on the solid support. The capture reagent is selected based on the type of analysis to be conducted. Capture reagents include but are not limited to aptamers, antibodies, antigens, adnectins, ankyrins, other antibody mimetics and other protein scaffolds, autoantibodies, chimeras, small molecules, an F(ab′)2 fragment, a single chain antibody fragment, an Fv fragment, a single chain Fv fragment, a nucleic acid, a lectin, a ligand-binding receptor, affybodies, nanobodies, imprinted polymers, avimers, peptidomimetics, a hormone receptor, a cytokine receptor, and synthetic receptors, and modifications and fragments of these.

In some embodiments, a biomarker value is detected using a biomarker/capture reagent complex. In other embodiments, the biomarker value is derived from the biomarker/capture reagent complex and is detected indirectly, such as, for example, as a result of a reaction that is subsequent to the biomarker/capture reagent interaction, but is dependent on the formation of the biomarker/capture reagent complex.

In some embodiments, the biomarker value is detected directly from the biomarker in a biological sample. In one embodiment, the biomarkers are detected using a multiplexed format that allows for the simultaneous detection of two or more biomarkers in a biological sample. In one embodiment of the multiplexed format, capture reagents are immobilized, directly or indirectly, covalently or non-covalently, in discrete locations on a solid support. In another embodiment, a multiplexed format uses discrete solid supports where each solid support has a unique capture reagent associated with that solid support, such as, for example quantum dots. In another embodiment, an individual device is used for the detection of each one of multiple biomarkers to be detected in a biological sample. Individual devices can be configured to permit each biomarker in the biological sample to be processed simultaneously. For example, a microtiter plate can be used such that each well in the plate is used to uniquely analyze one of multiple biomarkers to be detected in a biological sample.

In one or more of the foregoing embodiments, a fluorescent tag can be used to label a component of the biomarker/capture complex to enable the detection of the biomarker value. In various embodiments, the fluorescent label can be conjugated to a capture reagent specific to any of the biomarkers described herein using known techniques, and the fluorescent label can then be used to detect the corresponding biomarker value. Suitable fluorescent labels include rare earth chelates, fluorescein and its derivatives, rhodamine and its derivatives, dansyl, allophycocyanin, PBXL-3, Qdot 605, Lissamine, phycoerythrin, Texas Red, and other such compounds.

In one embodiment, the fluorescent label is a fluorescent dye molecule. In some embodiments, the fluorescent dye molecule includes at least one substituted indolium ring system in which the substituent on the 3-carbon of the indolium ring contains a chemically reactive group or a conjugated substance. In some embodiments, the dye molecule includes an AlexFluor molecule, such as, for example, AlexaFluor 488, AlexaFluor 532, AlexaFluor 647, AlexaFluor 680, or AlexaFluor 700. In other embodiments, the dye molecule includes a first type and a second type of dye molecule, such as, e.g., two different AlexaFluor molecules. In other embodiments, the dye molecule includes a first type and a second type of dye molecule, and the two dye molecules have different emission spectra.

Fluorescence can be measured with a variety of instrumentation compatible with a wide range of assay formats. For example, spectrofluorimeters have been designed to analyze microtiter plates, microscope slides, printed arrays, cuvettes, etc. See Principles of Fluorescence Spectroscopy, by J. R. Lakowicz, Springer Science+Business Media, Inc., 2004. See Bioluminescence & Chemiluminescence: Progress & Current Applications; Philip E. Stanley and Larry J. Kricka editors, World Scientific Publishing Company, January 2002.

In one or more of the foregoing embodiments, a chemiluminescence tag can optionally be used to label a component of the biomarker/capture complex to enable the detection of a biomarker value. Suitable chemiluminescent materials include any of oxalyl chloride, Rodamin 6G, Ru(bipy)32+, TMAE (tetrakis(dimethylamino) ethylene), Pyrogallol (1,2,3-trihydroxibenzene), Lucigenin, peroxyoxalates, Aryl oxalates, Acridinium esters, dioxetanes, and others.

In yet other embodiments, the detection method includes an enzyme/substrate combination that generates a detectable signal that corresponds to the biomarker value. Generally, the enzyme catalyzes a chemical alteration of the chromogenic substrate which can be measured using various techniques, including spectrophotometry, fluorescence, and chemiluminescence. Suitable enzymes include, for example, luciferases, luciferin, malate dehydrogenase, urease, horseradish peroxidase (HRPO), alkaline phosphatase, beta-galactosidase, glucoamylase, lysozyme, glucose oxidase, galactose oxidase, and glucose-6-phosphate dehydrogenase, uricase, xanthine oxidase, lactoperoxidase, microperoxidase, and the like.

In yet other embodiments, the detection method can be a combination of fluorescence, chemiluminescence, radionuclide or enzyme/substrate combinations that generate a measurable signal. Multimodal signaling could have unique and advantageous characteristics in biomarker assay formats.

More specifically, the biomarker values for the biomarkers described herein can be detected using known analytical methods including, singleplex aptamer assays, multiplexed aptamer assays, singleplex or multiplexed immunoassays, mRNA expression profiling, miRNA expression profiling, mass spectrometric analysis, histological/cytological methods, and the like, as detailed herein.

### Detection of Biomarkers Using In Vivo Molecular Imaging Technologies

Any of the described biomarkers may also be used in molecular imaging tests. For example, an imaging agent can be coupled to any of the described biomarkers, which can be used to aid in SCLC diagnosis, to monitor disease progression/remission or metastasis, to monitor for disease recurrence, or to monitor response to therapy, among other uses.

In vivo imaging technologies provide non-invasive methods for determining the state of a particular disease in the body of an individual. For example, entire portions of the body, or even the entire body, may be viewed as a three dimensional image, thereby providing valuable information concerning morphology and structures in the body. Such technologies may be combined with the detection of the biomarkers described herein to provide information concerning the cancer status, in particular the SCLC status, of an individual.

The use of in vivo molecular imaging technologies is expanding due to various advances in technology. These advances include the development of new contrast agents or labels, such as radiolabels and/or fluorescent labels, which can provide strong signals within the body; and the development of powerful new imaging technology, which can detect and analyze these signals from outside the body, with sufficient sensitivity and accuracy to provide useful information. The contrast agent can be visualized in an appropriate imaging system, thereby providing an image of the portion or portions of the body in which the contrast agent is located. The contrast agent may be bound to or associated with a capture reagent, such as an aptamer or an antibody, for example, and/or with a peptide or protein, or an oligonucleotide (for example, for the detection of gene expression), or a complex containing any of these with one or more macromolecules and/or other particulate forms.

The contrast agent may also feature a radioactive atom that is useful in imaging. Suitable radioactive atoms include technetium-99m or iodine-123 for scintigraphic studies. Other readily detectable moieties include, for example, spin labels for magnetic resonance imaging (MRI) such as, for example, iodine-123, iodine-131, indium-111, fluorine-19, carbon-13, nitrogen-15, oxygen-17, gadolinium, manganese, or iron. Such labels are well known in the art and could easily be selected by one of ordinary skill in the art.

Standard imaging techniques include but are not limited to magnetic resonance imaging, computed tomography scanning, positron emission tomography (PET), single photon emission computed tomography (SPECT), and the like. For diagnostic in vivo imaging, the type of detection instrument available is a major factor in selecting a given contrast agent, such as a given radionuclide and the particular biomarker that it is used to target (protein, mRNA, and the like). The radionuclide chosen typically has a type of decay that is detectable by a given type of instrument. Also, when selecting a radionuclide for in vivo diagnosis, its half-life should be long enough to enable detection at the time of maximum uptake by the target tissue but short enough that deleterious radiation of the host is minimized.

Exemplary imaging techniques include but are not limited to PET and SPECT, which are imaging techniques in which a radionuclide is synthetically or locally administered to an individual. The subsequent uptake of the radiotracer is measured over time and used to obtain information about the targeted tissue and the biomarker. Because of the high-energy (gamma-ray) emissions of the specific isotopes employed and the sensitivity and sophistication of the instruments used to detect them, the two-dimensional distribution of radioactivity may be inferred from outside of the body.

Commonly used positron-emitting nuclides in PET include, for example, carbon-11, nitrogen-13, oxygen-15, and fluorine-18. Isotopes that decay by electron capture and/or gamma-emission are used in SPECT and include, for example iodine-123 and technetium-99m. An exemplary method for labeling amino acids with technetium-99m is the reduction of pertechnetate ion in the presence of a chelating precursor to form the labile technetium-99m-precursor complex, which, in turn, reacts with the metal binding group of a bifunctionally modified chemotactic peptide to form a technetium-99m-chemotactic peptide conjugate.

Antibodies are frequently used for such in vivo imaging diagnostic methods. The preparation and use of antibodies for in vivo diagnosis is well known in the art. Labeled antibodies which specifically bind any of the biomarkers described herein can be injected into an individual suspected of having a certain type of cancer (e.g., SCLC), detectable according to the particular biomarker used, for the purpose of diagnosing or evaluating the disease status of the individual. The label used will be selected in accordance with the imaging modality to be used, as previously described. Localization of the label permits determination of the spread of the cancer. The amount of label within an organ or tissue also allows determination of the presence or absence of cancer in that organ or tissue.

Similarly, aptamers may be used for such in vivo imaging diagnostic methods. For example, an aptamer that was used to identify a particular biomarker described herein (and therefore binds specifically to that particular biomarker) may be appropriately labeled and injected into an individual suspected of having SCLC, detectable according to the particular biomarker, for the purpose of diagnosing or evaluating the SCLC status of the individual. The label used will be selected in accordance with the imaging modality to be used, as previously described. Localization of the label permits determination of the spread of the cancer. The amount of label within an organ or tissue also allows determination of the presence or absence of cancer in that organ or tissue. Aptamer-directed imaging agents could have unique and advantageous characteristics relating to tissue penetration, tissue distribution, kinetics, elimination, potency, and selectivity as compared to other imaging agents.

Such techniques may also optionally be performed with labeled oligonucleotides, for example, for detection of gene expression through imaging with antisense oligonucleotides. These methods are used for in situ hybridization, for example, with fluorescent molecules or radionuclides as the label. Other methods for detection of gene expression include, for example, detection of the activity of a reporter gene.

Another general type of imaging technology is optical imaging, in which fluorescent signals within the subject are detected by an optical device that is external to the subject. These signals may be due to actual fluorescence and/or to bioluminescence. Improvements in the sensitivity of optical detection devices have increased the usefulness of optical imaging for in vivo diagnostic assays.

The use of in vivo molecular biomarker imaging is increasing, including for clinical trials, for example, to more rapidly measure clinical efficacy in trials for new cancer therapies and/or to avoid prolonged treatment with a placebo for those diseases, such as multiple sclerosis, in which such prolonged treatment may be considered to be ethically questionable.

### Determination of Biomarker Values Using Histology/Cytology Methods

For evaluation of SCLC, a variety of tissue samples may be used in histological or cytological methods. Sample selection depends on the primary tumor location and sites of metastases. For example, endo- and trans-bronchial biopsies, fine needle aspirates, cutting needles, and core biopsies can be used for histology. Bronchial washing and brushing, pleural aspiration, pleural fluid, and sputum, can be used for cytology. While cytological analysis is still used in the diagnosis of SCLC, histological methods are known to provide better sensitivity for the detection of cancer. Any of the biomarkers identified herein that were shown to be up- or down-regulated in individuals with SCLC can be used to stain a histological specimen as an indication of disease.

In one embodiment, one or more capture reagents specific to the corresponding biomarker(s) are used in a cytological evaluation of a lung tissue cell sample and may include one or more of the following: collecting a cell sample, fixing the cell sample, dehydrating, clearing, immobilizing the cell sample on a microscope slide, permeabilizing the cell sample, treating for analyte retrieval, staining, destaining, washing, blocking, and reacting with one or more capture reagent/s in a buffered solution. In another embodiment, the cell sample is produced from a cell block.

In another embodiment, one or more capture reagent(s) specific to the corresponding biomarker(s) are used in a histological evaluation of a lung tissue sample and may include one or more of the following: collecting a tissue specimen, fixing the tissue sample, dehydrating, clearing, immobilizing the tissue sample on a microscope slide, permeabilizing the tissue sample, treating for analyte retrieval, staining, destaining, washing, blocking, rehydrating, and reacting with capture reagent(s) in a buffered solution. In another embodiment, fixing and dehydrating are replaced with freezing.

In another embodiment, the one or more aptamer(s) specific to the corresponding biomarker(s) are reacted with the histological or cytological sample and can serve as the nucleic acid target in a nucleic acid amplification method. Suitable nucleic acid amplification methods include, for example, PCR, q-beta replicase, rolling circle amplification, strand displacement, helicase dependent amplification, loop mediated isothermal amplification, ligase chain reaction, and restriction and circularization aided rolling circle amplification.

In one embodiment, the one or more capture reagent(s) specific to the corresponding biomarkers for use in the histological or cytological evaluation are mixed in a buffered solution that can include any of the following: blocking materials, competitors, detergents, stabilizers, carrier nucleic acid, polyanionic materials, and the like.

A “cytology protocol” generally includes sample collection, sample fixation, sample immobilization, and staining. “Cell preparation” can include several processing steps after sample collection, including the use of one or more slow off-rate aptamers for the staining of the prepared cells.

Sample collection can include directly placing the sample in an untreated transport container, placing the sample in a transport container containing some type of media, or placing the sample directly onto a slide (immobilization) without any treatment or fixation.

Sample immobilization can be improved by applying a portion of the collected specimen to a glass slide that is treated with polylysine, gelatin, or a silane. Slides can be prepared by smearing a thin and even layer of cells across the slide. Care is generally taken to minimize mechanical distortion and drying artifacts. Liquid specimens can be processed in a cell block method. Alternatively, liquid specimens can be mixed 1:1 with the fixative solution for about 10 minutes at room temperature.

Cell blocks can be prepared from residual effusions, sputum, urine sediments, gastrointestinal fluids, pulmonary fluids, cell scraping, or fine needle aspirates. Cells are concentrated or packed by centrifugation or membrane filtration. A number of methods for cell block preparation have been developed. Representative procedures include the fixed sediment, bacterial agar, or membrane filtration methods. In the fixed sediment method, the cell sediment is mixed with a fixative like Bouins, picric acid, or buffered formalin and then the mixture is centrifuged to pellet the fixed cells. The supernatant is removed, drying the cell pellet as completely as possible. The pellet is collected and wrapped in lens paper and then placed in a tissue cassette. The tissue cassette is placed in a jar with additional fixative and processed as a tissue sample. Agar method is very similar but the pellet is removed and dried on paper towel and then cut in half. The cut side is placed in a drop of melted agar on a glass slide and then the pellet is covered with agar making sure that no bubbles form in the agar. The agar is allowed to harden and then any excess agar is trimmed away. This is placed in a tissue cassette and the tissue process completed. Alternatively, the pellet may be directly suspended in 2% liquid agar at 65° C. and the sample centrifuged. The agar cell pellet is allowed to solidify for an hour at 4° C. The solid agar may be removed from the centrifuge tube and sliced in half The agar is wrapped in filter paper and then the tissue cassette. Processing from this point forward is as described above. Centrifugation can be replaced in any these procedures with membrane filtration. Any of these processes may be used to generate a “cell block sample.”

Cell blocks can be prepared using specialized resin including Lowicryl resins, LR White, LR Gold, Unicryl, and MonoStep. These resins have low viscosity and can be polymerized at low temperatures and with ultra violet (UV) light. The embedding process relies on progressively cooling the sample during dehydration, transferring the sample to the resin, and polymerizing a block at the final low temperature at the appropriate UV wavelength.

Cell block sections can be stained with hematoxylin-eosin for cytomorphological examination while additional sections are used for examination for specific markers.

Whether the process is cytological or histological, the sample may be fixed prior to additional processing to prevent sample degradation. This process is called “fixation” and describes a wide range of materials and procedures that may be used interchangeably. The sample fixation protocol and reagents are best selected empirically based on the targets to be detected and the specific cell/tissue type to be analyzed. Sample fixation relies on reagents such as ethanol, polyethylene glycol, methanol, formalin, or isopropanol. The samples should be fixed as soon after collection and affixation to the slide as possible. However, the fixative selected can introduce structural changes into various molecular targets making their subsequent detection more difficult. The fixation and immobilization processes and their sequence can modify the appearance of the cell and these changes must be anticipated and recognized by the cytotechnologist. Fixatives can cause shrinkage of certain cell types and cause the cytoplasm to appear granular or reticular. Many fixatives function by crosslinking cellular components. This can damage or modify specific epitopes, generate new epitopes, cause molecular associations, and reduce membrane permeability. Formalin fixation is one of the most common cytological/histological approaches. Formalin forms methyl bridges between neighboring proteins or within proteins. Precipitation or coagulation is also used for fixation and ethanol is frequently used in this type of fixation. A combination of crosslinking and precipitation can also be used for fixation. A strong fixation process is best at preserving morphological information while a weaker fixation process is best for the preservation of molecular targets.

A representative fixative is 50% absolute ethanol, 2 mM polyethylene glycol (PEG), 1.85% formaldehyde. Variations on this formulation include ethanol (50% to 95%), methanol (20%-50%), and formalin (formaldehyde) only. Another common fixative is 2% PEG 1500, 50% ethanol, and 3% methanol. Slides are place in the fixative for about 10 to 15 minutes at room temperature and then removed and allowed to dry. Once slides are fixed they can be rinsed with a buffered solution like PBS.

A wide range of dyes can be used to differentially highlight and contrast or “stain” cellular, sub-cellular, and tissue features or morphological structures. Hematoylin is used to stain nuclei a blue or black color. Orange G-6 and Eosin Azure both stain the cell's cytoplasm. Orange G stains keratin and glycogen containing cells yellow. Eosin Y is used to stain nucleoli, cilia, red blood cells, and superficial epithelial squamous cells. Romanowsky stains are used for air dried slides and are useful in enhancing pleomorphism and distinguishing extracellular from intracytoplasmic material.

The staining process can include a treatment to increase the permeability of the cells to the stain. Treatment of the cells with a detergent can be used to increase permeability. To increase cell and tissue permeability, fixed samples can be further treated with solvents, saponins, or non-ionic detergents. Enzymatic digestion can also improve the accessibility of specific targets in a tissue sample.

After staining, the sample is dehydrated using a succession of alcohol rinses with increasing alcohol concentration. The final wash is done with xylene or a xylene substitute, such as a citrus terpene, that has a refractive index close to that of the coverslip to be applied to the slide. This final step is referred to as clearing. Once the sample is dehydrated and cleared, a mounting medium is applied. The mounting medium is selected to have a refractive index close to the glass and is capable of bonding the coverslip to the slide. It will also inhibit the additional drying, shrinking, or fading of the cell sample.

Regardless of the stains or processing used, the final evaluation of the lung cytological specimen is made by some type of microscopy to permit a visual inspection of the morphology and a determination of the marker's presence or absence. Exemplary microscopic methods include brightfield, phase contrast, fluorescence, and differential interference contrast.

If secondary tests are required on the sample after examination, the coverslip may be removed and the slide destained. Destaining involves using the original solvent systems used in staining the slide originally without the added dye and in a reverse order to the original staining procedure. Destaining may also be completed by soaking the slide in an acid alcohol until the cells are colorless. Once colorless the slides are rinsed well in a water bath and the second staining procedure applied.

In addition, specific molecular differentiation may be possible in conjunction with the cellular morphological analysis through the use of specific molecular reagents such as antibodies or nucleic acid probes or aptamers. This improves the accuracy of diagnostic cytology. Micro-dissection can be used to isolate a subset of cells for additional evaluation, in particular, for genetic evaluation of abnormal chromosomes, gene expression, or mutations.

Preparation of a tissue sample for histological evaluation involves fixation, dehydration, infiltration, embedding, and sectioning. The fixation reagents used in histology are very similar or identical to those used in cytology and have the same issues of preserving morphological features at the expense of molecular ones such as individual proteins. Time can be saved if the tissue sample is not fixed and dehydrated but instead is frozen and then sectioned while frozen. This is a more gentle processing procedure and can preserve more individual markers. However, freezing is not acceptable for long term storage of a tissue sample as subcellular information is lost due to the introduction of ice crystals. Ice in the frozen tissue sample also prevents the sectioning process from producing a very thin slice and thus some microscopic resolution and imaging of subcellular structures can be lost. In addition to formalin fixation, osmium tetroxide is used to fix and stain phospholipids (membranes).

Dehydration of tissues is accomplished with successive washes of increasing alcohol concentration. Clearing employs a material that is miscible with alcohol and the embedding material and involves a stepwise process starting at 50:50 alcohol clearing reagent and then 100% clearing agent (xylene or xylene substitute). Infiltration involves incubating the tissue with a liquid form of the embedding agent (warm wax, nitrocellulose solution) first at 50:50 embedding agent/clearing agent and the 100% embedding agent. Embedding is completed by placing the tissue in a mold or cassette and filling with melted embedding agent such as wax, agar, or gelatin. The embedding agent is allowed to harden. The hardened tissue sample may then be sliced into thin section for staining and subsequent examination.

Prior to staining, the tissue section is dewaxed and rehydrated. Xylene is used to dewax the section, one or more changes of xylene may be used, and the tissue is rehydrated by successive washes in alcohol of decreasing concentration. Prior to dewax, the tissue section may be heat immobilized to a glass slide at about 80° C. for about 20 minutes.

Laser capture micro-dissection allows the isolation of a subset of cells for further analysis from a tissue section.

As in cytology, to enhance the visualization of the microscopic features, the tissue section or slice can be stained with a variety of stains. A large menu of commercially available stains can be used to enhance or identify specific features.

To further increase the interaction of molecular reagents with cytological/histological samples, a number of techniques for “analyte retrieval” have been developed. The first such technique uses high temperature heating of a fixed sample. This method is also referred to as heat-induced epitope retrieval or HIER. A variety of heating techniques have been used, including steam heating, microwaving, autoclaving, water baths, and pressure cooking, or a combination of these methods of heating. Analyte retrieval solutions include, for example, water, citrate, and normal saline buffers. The key to analyte retrieval is the time at high temperature but lower temperatures for longer times have also been successfully used. Another key to analyte retrieval is the pH of the heating solution. Low pH has been found to provide the best immunostaining but also gives rise to backgrounds that frequently require the use of a second tissue section as a negative control. The most consistent benefit (increased immunostaining without increase in background) is generally obtained with a high pH solution regardless of the buffer composition. The analyte retrieval process for a specific target is empirically optimized for the target using heat, time, pH, and buffer composition as variables for process optimization. Using the microwave analyte retrieval method allows for sequential staining of different targets with antibody reagents. The time required to achieve antibody and enzyme complexes between staining steps has also been shown to degrade cell membrane analytes. Microwave heating methods have improved in situ hybridization methods as well.

To initiate the analyte retrieval process, the section is first dewaxed and hydrated. The slide is then placed in 10 mM sodium citrate buffer pH 6.0 in a dish or jar. A representative procedure uses an 1100W microwave and microwaves the slide at 100% power for 2 minutes followed by microwaving the slides using 20% power for 18 minutes after checking to be sure the slide remains covered in liquid. The slide is then allowed to cool in the uncovered container and then rinsed with distilled water. HIER may be used in combination with an enzymatic digestion to improve the reactivity of the target to immunochemical reagents.

One such enzymatic digestion protocol uses proteinase K. A 20 g/mL concentration of proteinase K is prepared in 50 mM Tris Base, 1 mM EDTA, 0.5% Triton X-100, pH 8.0 buffer. The process first involves dewaxing sections in two changes of xylene, 5 minutes each. Then the sample is hydrated in two changes of 100% ethanol for 3 minutes each, 95% and 80% ethanol for 1 minute each, and then rinsed in distilled water. Sections are covered with Proteinase K working solution and incubated 10-20 minutes at 37° C. in humidified chamber (optimal incubation time may vary depending on tissue type and degree of fixation). The sections are cooled at room temperature for 10 minutes and then rinsed in PBS Tween 20 for 2×2 min. If desired, sections can be blocked to eliminate potential interference from endogenous compounds and enzymes. The section is then incubated with primary antibody at appropriate dilution in primary antibody dilution buffer for 1 hour at room temperature or overnight at 4° C. The section is then rinsed with PBS Tween 20 for 2×2 min. Additional blocking can be performed, if required for the specific application, followed by additional rinsing with PBS Tween 20 for 3×2 min and then finally the immunostaining protocol completed.

A simple treatment with 1% SDS at room temperature has also been demonstrated to improve immunohistochemical staining. Analyte retrieval methods have been applied to slide mounted sections as well as free floating sections. Another treatment option is to place the slide in a jar containing citric acid and 0.1 Nonident P40 at pH 6.0 and heating to 95° C. The slide is then washed with a buffer solution like PBS.

For immunological staining of tissues it may be useful to block non-specific association of the antibody with tissue proteins by soaking the section in a protein solution like serum or non-fat dry milk.

Blocking reactions may include the need to reduce the level of endogenous biotin; eliminate endogenous charge effects; inactivate endogenous nucleases; and/or inactivate endogenous enzymes like peroxidase and alkaline phosphatase. Endogenous nucleases may be inactivated by degradation with proteinase K, by heat treatment, use of a chelating agent such as EDTA or EGTA, the introduction of carrier DNA or RNA, treatment with a chaotrope such as urea, thiourea, guanidine hydrochloride, guanidine thiocyanate, lithium perchlorate, and the like, or diethyl pyrocarbonate. Alkaline phosphatase may be inactivated by treated with 0.1 N HCl for 5 minutes at room temperature or treatment with 1 mM levamisole. Peroxidase activity may be eliminated by treatment with 0.03% hydrogen peroxide. Endogenous biotin may be blocked by soaking the slide or section in an avidin (streptavidin, neutravidin may be substituted) solution for at least 15 minutes at room temperature. The slide or section is then washed for at least 10 minutes in buffer. This may be repeated at least three times. Then the slide or section is soaked in a biotin solution for 10 minutes. This may be repeated at least three times with a fresh biotin solution each time. The buffer wash procedure is repeated. Blocking protocols should be minimized to prevent damaging either the cell or tissue structure or the target or targets of interest but one or more of these protocols could be combined to “block” a slide or section prior to reaction with one or more slow off-rate aptamers. See Basic Medical Histology: the Biology of Cells, Tissues and Organs, authored by Richard G. Kessel, Oxford University Press, 1998.

### Determination of Biomarker Values Using Mass Spectrometry Methods

A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Differences in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer, and time-of-flight mass analyzer. Additional mass spectrometry methods are well known in the art (see Burlingame et al. Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS)N, atmospheric pressure photoionization mass spectrometry (APPI-MS), APPI-MS/MS, and APPI-(MS)N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)2 fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g., diabodies) imprinted polymers, avimers, peptidomimetics, peptoids, peptide nucleic acids, threose nucleic acid, a hormone receptor, a cytokine receptor, and synthetic receptors, and modifications and fragments of these.

### Determination of Biomarker Values Using a Proximity Ligation Assay

A proximity ligation assay can be used to determine biomarker values. Briefly, a test sample is contacted with a pair of affinity probes that may be a pair of antibodies or a pair of aptamers, with each member of the pair extended with an oligonucleotide. The targets for the pair of affinity probes may be two distinct determinates on one protein or one determinate on each of two different proteins, which may exist as homo- or hetero-multimeric complexes. When probes bind to the target determinates, the free ends of the oligonucleotide extensions are brought into sufficiently close proximity to hybridize together. The hybridization of the oligonucleotide extensions is facilitated by a common connector oligonucleotide which serves to bridge together the oligonucleotide extensions when they are positioned in sufficient proximity. Once the oligonucleotide extensions of the probes are hybridized, the ends of the extensions are joined together by enzymatic DNA ligation.

Each oligonucleotide extension comprises a primer site for PCR amplification. Once the oligonucleotide extensions are ligated together, the oligonucleotides form a continuous DNA sequence which, through PCR amplification, reveals information regarding the identity and amount of the target protein, as well as, information regarding protein-protein interactions where the target determinates are on two different proteins. Proximity ligation can provide a highly sensitive and specific assay for real-time protein concentration and interaction information through use of real-time PCR. Probes that do not bind the determinates of interest do not have the corresponding oligonucleotide extensions brought into proximity and no ligation or PCR amplification can proceed, resulting in no signal being produced.

The foregoing assays enable the detection of biomarker values that are useful in methods described herein, where the methods comprise detecting, in a biological sample from an individual, at least N biomarker values that each correspond to a biomarker selected from the group consisting of the biomarkers provided herein, wherein a classification, as described in detail below, using the biomarker values indicates whether the individual has SCLC or whether the individual is likely to benefit from PARP inhibitor chemotherapy. While certain of the described SCLC biomarkers are useful alone for assigning a subject to receive PARP inhibitor chemotherapy, they are also useful for detecting and diagnosing SCLC, alone or in combination as multiple subsets of the SCLC biomarkers that are each useful as a panel of two or more biomarkers. Thus, various embodiments of the instant application provide combinations comprising one or more biomarkers as described herein. In other embodiments, N is selected to be any number from 1-10 biomarkers. It will be appreciated that N can be selected to be any number from any of the above described ranges, as well as similar, but higher order, ranges. In accordance with any of the methods described herein, biomarker values can be detected and classified individually or they can be detected and classified collectively, as for example in a multiplex assay format.

In another aspect, methods are provided for detecting an increased likelihood of sensitivity to a PARP inhibitor, or the presence or absence of SCLC, the methods comprising detecting, in a biological sample from an individual, at least N biomarker values that each correspond to a biomarker selected from the group consisting of the biomarkers provided herein, wherein a classification, as described in detail below, of the biomarker values indicates an absence of SCLC in the individual.

Except as otherwise noted, the methods and techniques of the present embodiments are generally performed according to conventional methods well known in the art and as described in various general and more specific references that are cited and discussed throughout the present specification.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables are specifically embraced by the present invention and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present invention and are disclosed herein just as if each and every such sub-combination of chemical groups was individually and explicitly disclosed herein.

In some embodiments, the test sample may be obtained from lung tissue, bronchial biopsy, sputum, and/or blood serum.

Those skilled in the art will recognize that the species listed or illustrated herein are not exhaustive, and that additional species within the scope of these defined terms may also be selected.

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the embodiments include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²I, respectively.

A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

### Pharmaceutical Compositions and Methods of Treatment

For treatment purposes, pharmaceutical compositions comprising the compounds described herein may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the invention are sterile compositions. Pharmaceutical compositions may be prepared using compounding techniques known or that become available to those skilled in the art.

Sterile compositions are also contemplated by the invention, including compositions that are in accord with national and local regulations governing such compositions.

The pharmaceutical compositions and compounds described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the invention may be administered by a suitable route of delivery, such as oral, parenteral, rectal, nasal, topical, or ocular routes, or by inhalation. Preferably, the compositions are formulated for intravenous or oral administration.

For oral administration, the PARP inhibitor or talazoparib may be provided in a solid form, such as a tablet or capsule, or as a solution, emulsion, or suspension. To prepare the oral compositions, the active agent may be formulated to yield a dosage of, e.g., from about 0.01 to about 50 mg/kg daily, or from about 0.05 to about 20 mg/kg daily, or from about 0.1 to about 10 mg/kg daily. In some embodiments the oral dosage form provides a dose of about 25 to about 1100 μg/day, or about 0.5 to about 2 mg per day, or of about 1 mg/day, or about 0.10 to 0.75 mg/kg/day, or about 0.25-0.30 mg/kg/day. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The inventive compositions may be formulated for rectal administration as a suppository. For parenteral use, including intravenous, intramuscular, intraperitoneal, intranasal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For nasal, inhaled, or oral administration, the inventive pharmaceutical compositions may be administered using, for example, a spray formulation also containing a suitable carrier.

For topical applications, the compounds of the present invention are preferably formulated as creams or ointments or a similar vehicle suitable for topical administration. For topical administration, the inventive compounds may be mixed with a pharmaceutical carrier at a concentration of about 0.1% to about 10% of drug to vehicle. Another mode of administering the agents of the invention may utilize a patch formulation to effect transdermal delivery.

As used herein, the terms “treat,” “treating,” and “treatment” refer to an approach for obtaining beneficial or desired results, including clinical results. For purposes of this invention, beneficial or desired results include, but are not limited to, alleviation of a symptom and/or diminishment of the extent of a symptom and/or preventing a worsening of a symptom associated with a disease or condition and/or reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder. In one variation, treatment of SCLC is indicated by, for example, a reduction in tumor size, slowing of tumor growth, or reduction in metastasis.

In treatment methods according to the invention, an “effective amount” means an amount or dose sufficient to generally bring about the desired therapeutic benefit in subjects needing such treatment. Effective amounts or doses of the compounds of the invention may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about 1 μg to 2 mg of active agent per kilogram of subject's body weight per day, preferably about 0.05 to 100 mg/kg/day, or about 1 to 35 mg/kg/day, or about 0.1 to 10 mg/kg/day. The total dosage may be given in single or divided dosage units (e.g., BID, TID, QID). In some embodiments, doses are from about 0.01 to about 50 mg/kg daily, or from about 0.05 to about 20 mg/kg daily, or from about 0.1 to about 10 mg/kg daily. In some embodiments the dosage form provides a dose of about 25 to about 1100 μg/day, or about 0.5 to about 2 mg per day, or of about 1 mg/day, or about 0.10 to 0.75 mg/kg/day, or about 0.25-0.30 mg/kg/day. In some embodiments, the total daily dose is administered in a single dose, or a single oral dose.

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms. Patients may also require chronic treatment on a long-term basis.

### EXAMPLES

The examples described herein are provided solely to illustrate representative embodiments of the invention. Accordingly, it should be understood that the invention is not to be limited to the specific conditions or details described in these or any other examples discussed herein, and that such examples are not to be construed as limiting the scope of the invention in any way. The following examples are provided to illustrate but not to limit the invention.

### Example 1: Cell Line Cytotoxicity Assay with Single Agent Talazoparib

Various SCLC cell lines (38) were obtained from ATCC (American Type Culture Collection), ECACC (European Collection of Cell Cultures), JCRB (Japanese Collection of Research Bioresources), and CLS Cell Lines Service as shown in Table 1.

TABLE 1

| Name | Vendor | Cat# | Name | Vendor | Cat# | Name | Vendor | Cat# |
|---|---|---|---|---|---|---|---|---|
| COR-L88 | ECACC | 92031917 | NCI-H211 | ATCC | CRL-5824 | NCI-H1618 | ATCC | CRL-5879 |
| SBC-5 | JCRB | JCRB0819 | NCI-H2141 | ATCC | CRL-5927 | NCI-H1694 | ATCC | CRL-5888 |
| DMS 114 | ATCC | CRL-2066 | NCI-H2171 | ATCC | CRL-5929 | NCI-H1930 | ATCC | CRL-5906 |
| DMS 79 | ATCC | CRL-2049 | NCI-H446 | ATCC | HTB-171 | NCI-H2081 | ATCC | CRL-5920 |
| NCI-H1836 | ATCC | CRL-5898 | NCI-H82 | ATCC | HTB-175 | SCLC-21H | CLS | 300225 |
| NCI-H1876 | ATCC | CRL-5902 | NCI-H889 | ATCC | CRL-5817 | NCI-H524 | ATCC | CRL-5831 |
| NCI-H1963 | ATCC | CRL-5982 | SHP-77 | ATCC | CRL-2195 | NCI-H526 | ATCC | CRL-5811 |
| NCI-H69 | ATCC | HTB-119 | NCI-H1105 | ATCC | CRL-5856 | NCI-H841 | ATCC | CRL-5845 |
| NCI-H1048 | ATCC | CRL-5853 | NCI-H2066 | ATCC | CRL-5917 | NCI-H2107 | ATCC | CRL-5983 |
| NCI-H1341 | ATCC | CRL-5864 | COR-L279 | ECACC | 96020724 | NCI-H748 | ATCC | CRL-5841 |

| | | | | | |
|---|---|---|---|---|---|
| NCI-H146 | ATCC | HTB-173 | DMS-153 | ATCC | CRL-2064 |
| NCI-H196 | ATCC | CRL-5823 | DMS-53 | ATCC | CRL-2062 |
| NCI-H2029 | ATCC | CRL-5913 | NCI-H1092 | ATCC | CRL-5855 |
| NCI-H209 | ATCC | HTB-172 | NCI-H1436 | ATCC | CRL-5871 |

Cells were grown in suggested media and seeded in 96 well plates at a pre-determined cell density. After 24 h, talazoparib at 2000, 400, 80, 16, 3.2, or 0.64 nM in 0.2% DMSO, or cisplatin at 100,000, 2000, 400, 80, 16, or 3.2 nM were added in duplicate, and incubated for an additional 5 or 7 days. Cell survival was determined by CellTiter Glo assay (Promega). Cell growth inhibition was calculated by two methods: (a) the treated cell counts relative to untreated control to obtain IC₅₀ (convention survival fraction method), or (b) doublings from baseline under treatment relative to doublings from baseline without treatment to obtain GI₅₀ (generational method), using GraphPad Prism5. Maximum inhibition levels for each method were also obtained.

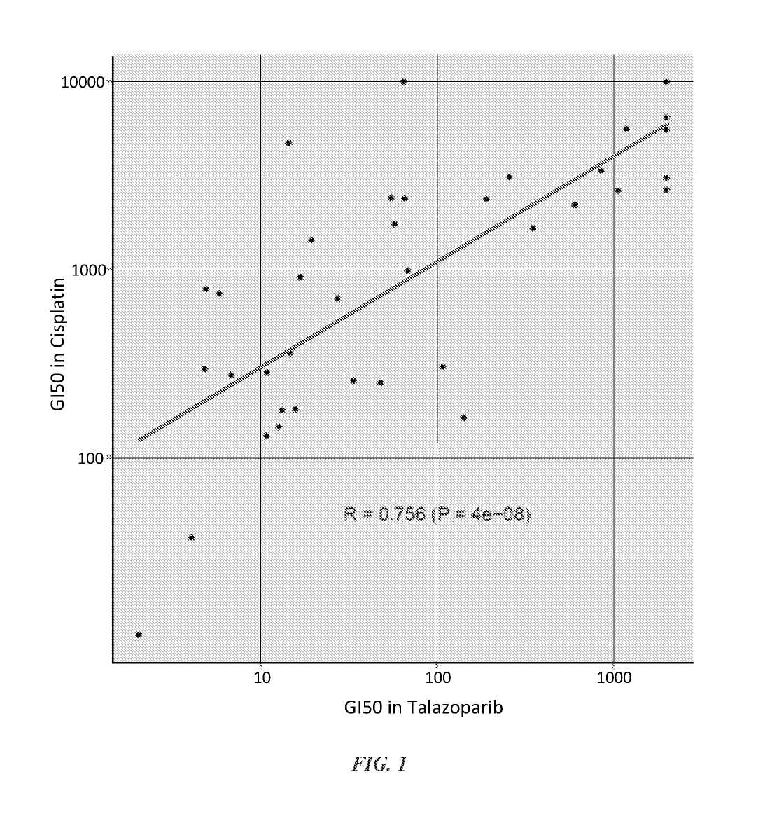

The 38 SCLC lines showed a wide range of sensitivities to talazoparib (GI₅₀ ranging from 2 nM to >2000 nM, with median GI₅₀=56 nM) and cisplatin treatment (GI₅₀ ranging from 10 nM to >10,000 nM). As shown in FIG. 1, sensitivity towards talazoparib and cisplatin are well correlated (Spearman correlation=0.756).

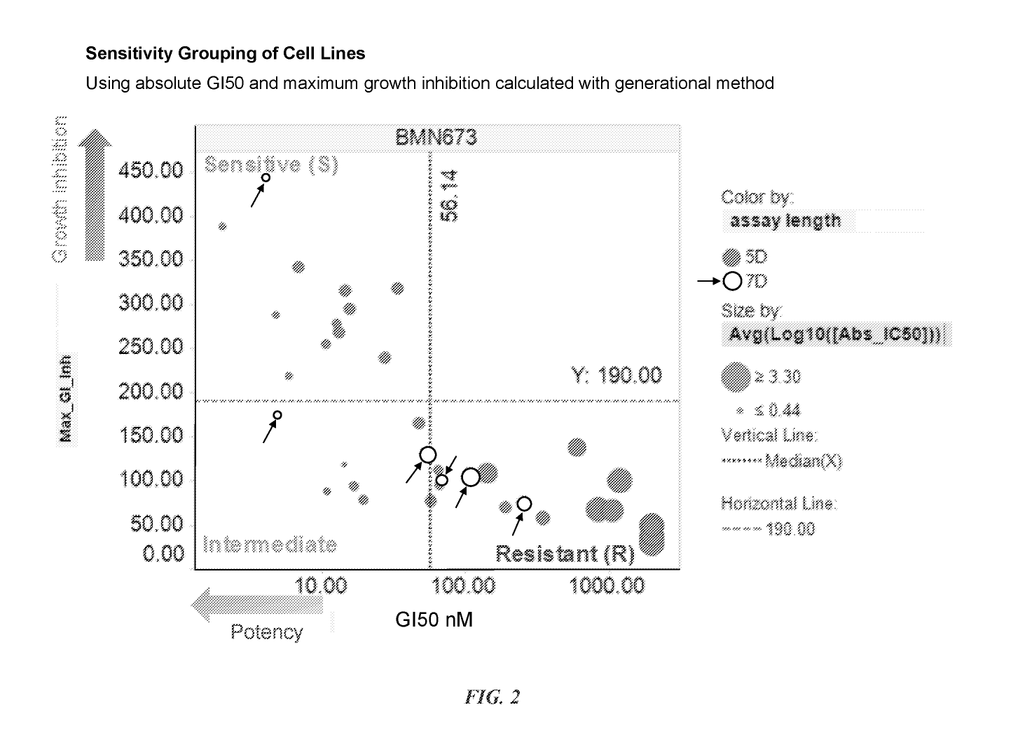

As shown in FIG. 2, in order to identify gene expression features associated with cell line sensitivity to talazoparib, cell lines were categorized into sensitivity groups based on their median GI₅₀ and 90% experimental maximum GI inhibition by talazoparib using the following criteria: Sensitive: maximum GI inhibition>190 and GI₅₀<56 nM (mean across SCLC cell lines screened); Resistant: maximum GI inhibition<190 and GI₅₀>56 nM, and the remaining cell lines as intermediate.

Gene expression data for SCLC cell lines were obtained from the CCLE portal (CCLE_Expression_Entrez_2012-09-29.gct; see Barretina Caponigro Stransky et al., Nature 483, 603-307, 2012). The average SLFN11 expression level for 36 SCLC cell lines was 5.78. The 16 cell lines with SLFN11 expression greater than 6.0 were labeled as the high SLFN11 group (6.4 to 9.5), and the remaining 20 SCLC cell lines were labeled as the low SLFN11 group (3.6 to 5.0).

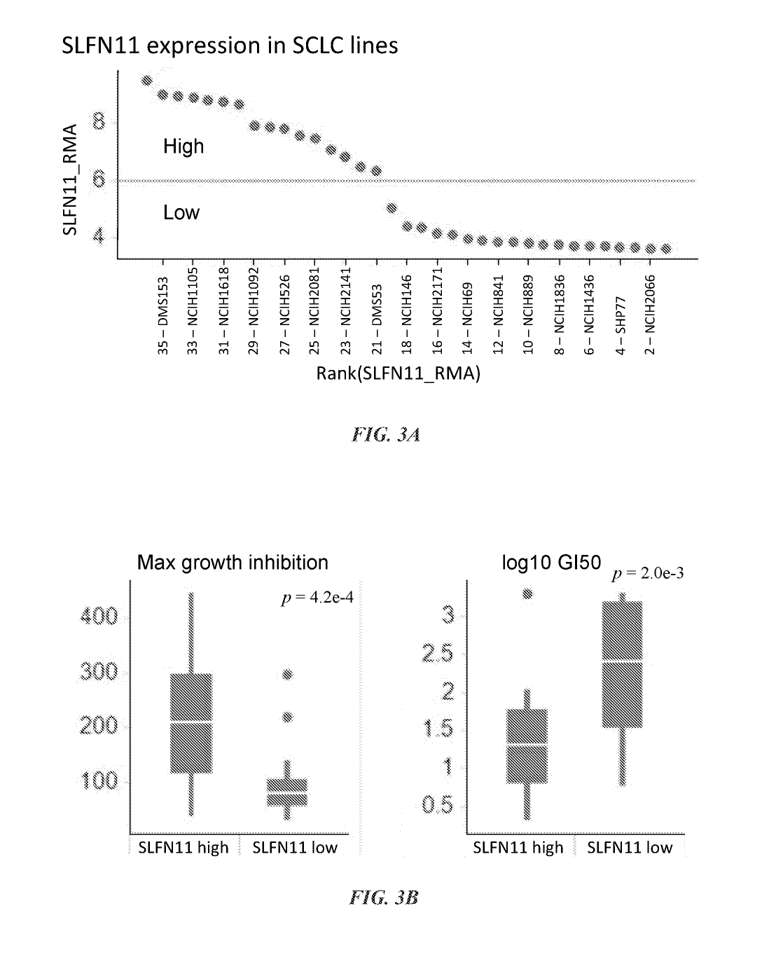

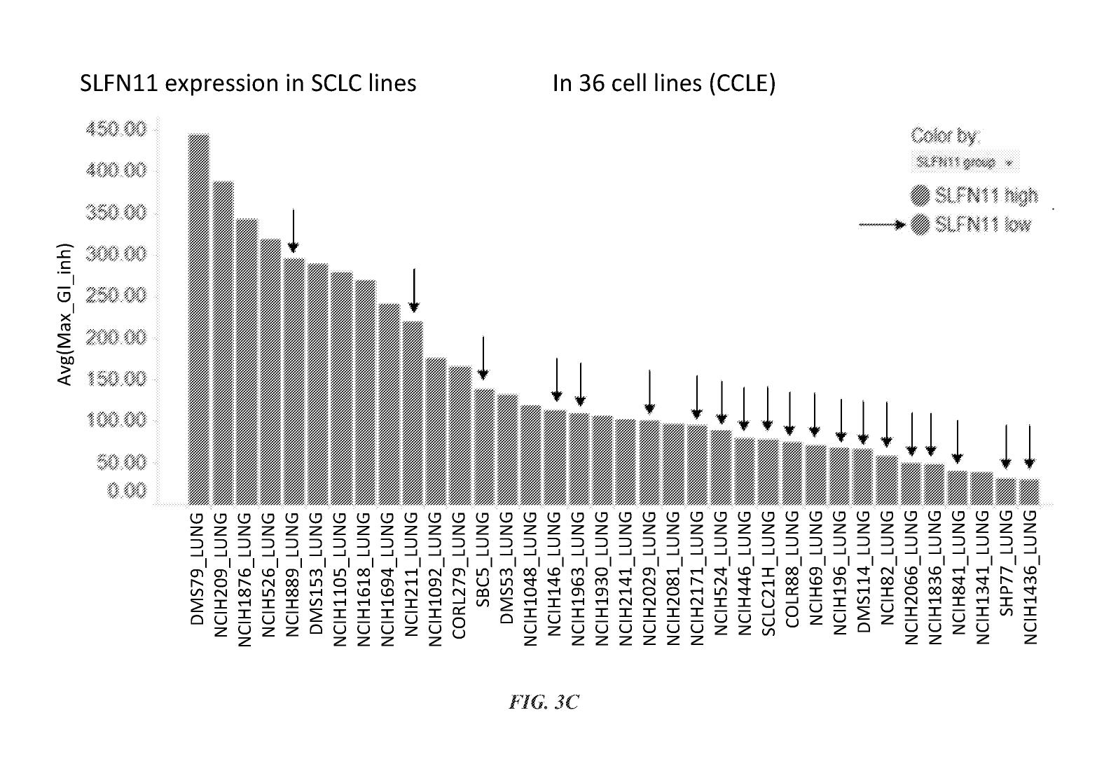

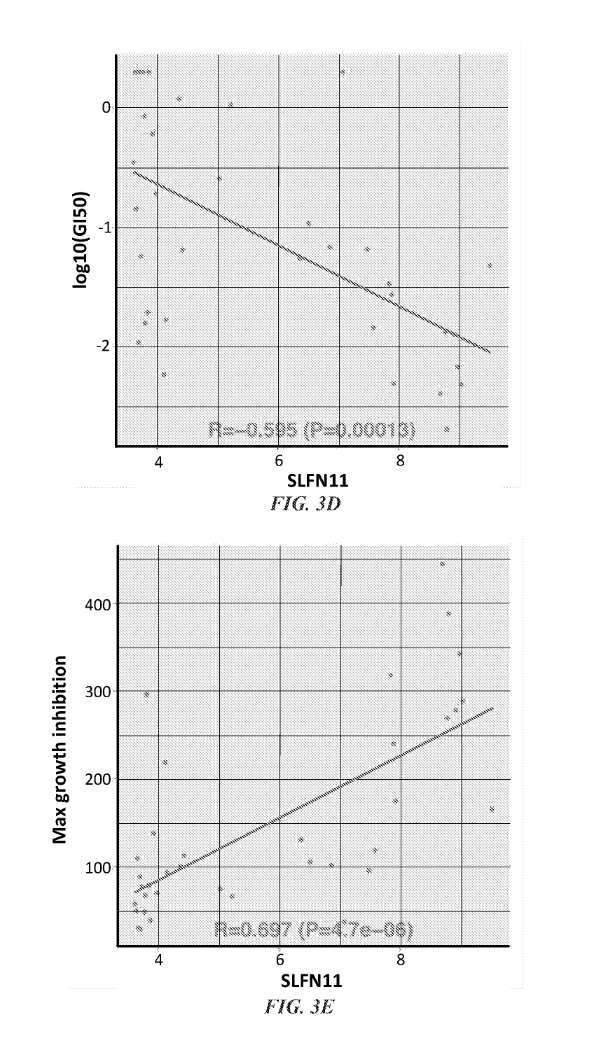

Standard statistical analyses, including a Spearman correlation and an ANOVA test, were applied to the resulting data. Differentially expressed genes between sensitive and resistant cell line groups were identified by the limma package in R (see Ritchie et al., Nucleic Acids Res. 2015, 43(7): e47). The moderated t-test using the limma package in R was used for differential gene expression analysis between the sensitive and resistant cell line groups, and the nominal p-value was adjusted for multiple hypothesis testing using the FDR method in R. SLFN11 was the most significant feature based on this analysis with adjusted p-value<0.5 and a nominal p-value of 2.3×10⁻⁵. Differential gene expression analysis based on sensitivity to talazoparib identified SLFN11 as the top gene expression feature as shown in FIGS. 3A-3E.

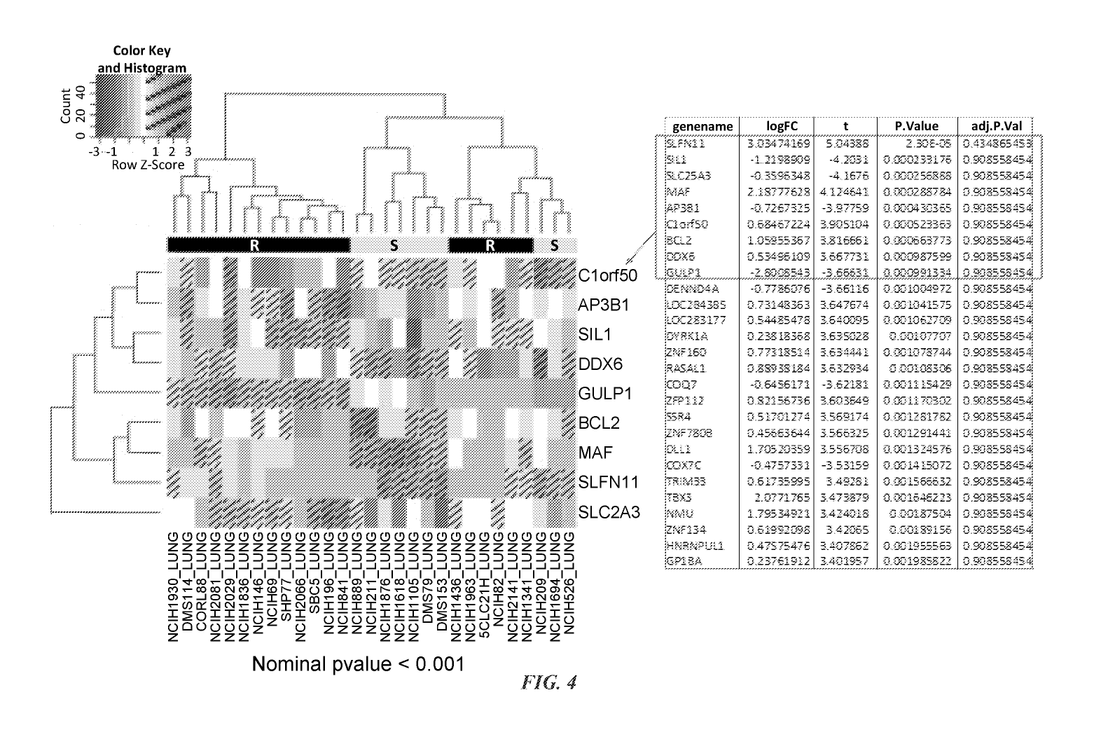

Shown in FIG. 4 are the top gene expression features associated with sensitivity, and those with nominal p-values<0.001 are highlighted in the box and were plotted by heatmap on left which showed a hierarchical clustering using the top nine genes. The nine identified genes included SLFN11, as well as genes involved in apoptosis regulation (BCL2, GULP1), oncogene (MAF), DNA/RNA regulation (DDX6), unfolded protein response (SIL1), organelle biogenesis (AP3B1), and phosphate transport (SLC25A3), and genes of unknown function (C1orf50), that were all nominally significant in association with cell line sensitivity to talazoparib.

Cell lysates extracted from 12 SCLC lines were subjected to Western blotting using SLFN11 antibody; β-tubulin was used as a loading control. As shown in FIG. 5, SLFN11 protein levels were well correlated with gene expression RMA data from the CCLE database, suggesting SLFN11protein expression is controlled epigenetically through transcription, likely by promotor methylation.

### Example 2: Cell Line-Derived Xenograft Models

Human NCI-H1048, NCI-H209, and NCI-H69 SCLC tumor cells were injected subcutaneously in the flanks of BALB/c nude mice. When tumors reached approximately 130 mm³ average volume, animals (n=8 per group) were treated with vehicle (Q1D×28, p.o.), cisplatin (6 mg/kg, Q6D×2 i.p.), or talazoparib (0.33 mg/kg, Q1D×28 p.o.). Tumor growth and animal body weight were monitored twice per week by standard methods.

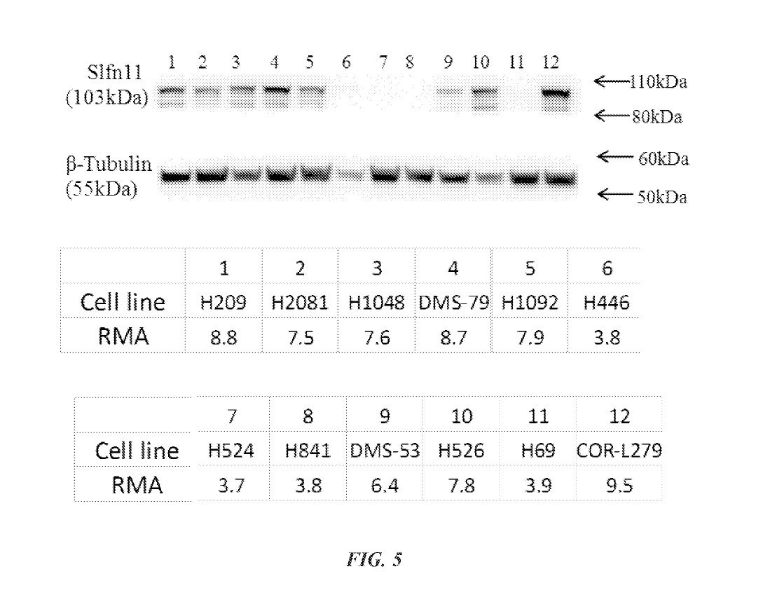

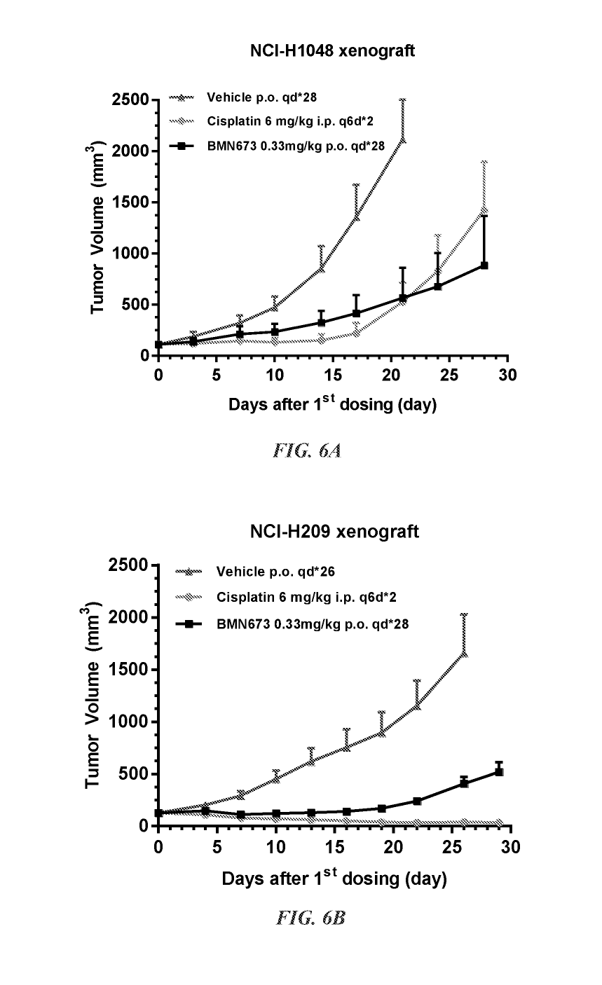

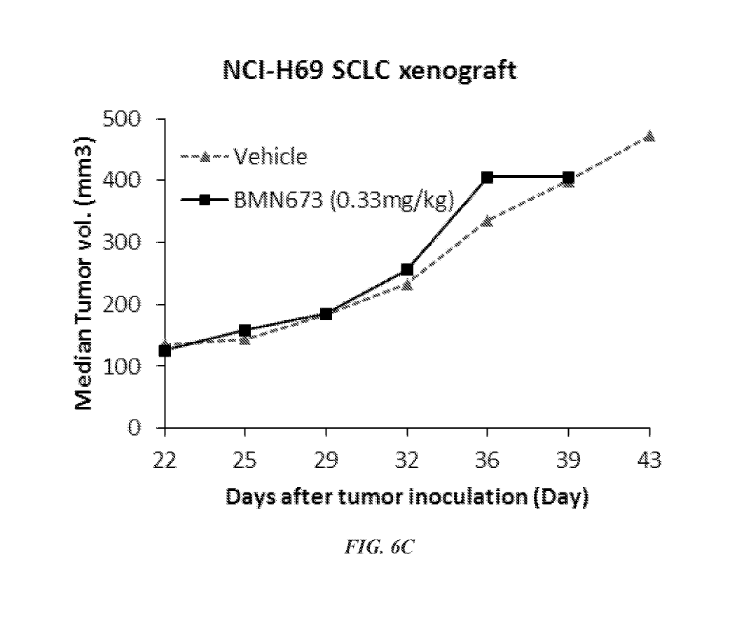

High SLFN11-expressing SCLC xenograft models NCI-H1048 (FIG. 6A) and NCI-H209 (FIG. 6B) as well as low SLFN11-expressing model NCI-H69 (FIG. 6C) were evaluated for their responsiveness to talazoparib single agent treatment. Tumor growth data confirmed that the H209 and H1048 models are much more sensitive to talazoparib than the H69 model under similar experimental conditions and that the response is correlated with RMA levels (Table 2).

TABLE 2

BMN 673 Sensitivity

| SCLC | SLFN11 | In vitro | In vivo |
|---|---|---|---|
| Cell line | RMA | GI₅₀ (nM) | Tumor growth |
| NCI-H209 | 8.794 | 2.03 | Delay |
| NCI-H1048 | 7.583 | 14.4 | Delay |
| NCI-H69 | 3.981 | 190.2 | No delay |

### Example 3: Human Patient-Derived Xenograft (PDX) Model

Twelve human SCLC PDX models (obtained from Crown Biosciences, OncoTest, WuxiAppTec) were evaluated for their responses to talazoparib single agent treatment. PDX tumors were propagated subcutaneously in immunocompromised mice at passage 3 to 13. When tumors reached approximately 150 mm³ average volume, animals (n=5 per group) were administered orally with vehicle (once daily dose), or talazoparib at the maximum tolerated dose (MTD; 0.25-0.3 mg/kg, once daily). Tumor volume and animal body weight were measured twice weekly until the end of study or until tumor size exceeded 2000 mm³. Untreated tumor samples were collected from mice. Median tumor volume on Day 21 and beyond after first treatment was used to calculate the change from baseline to evaluate response.

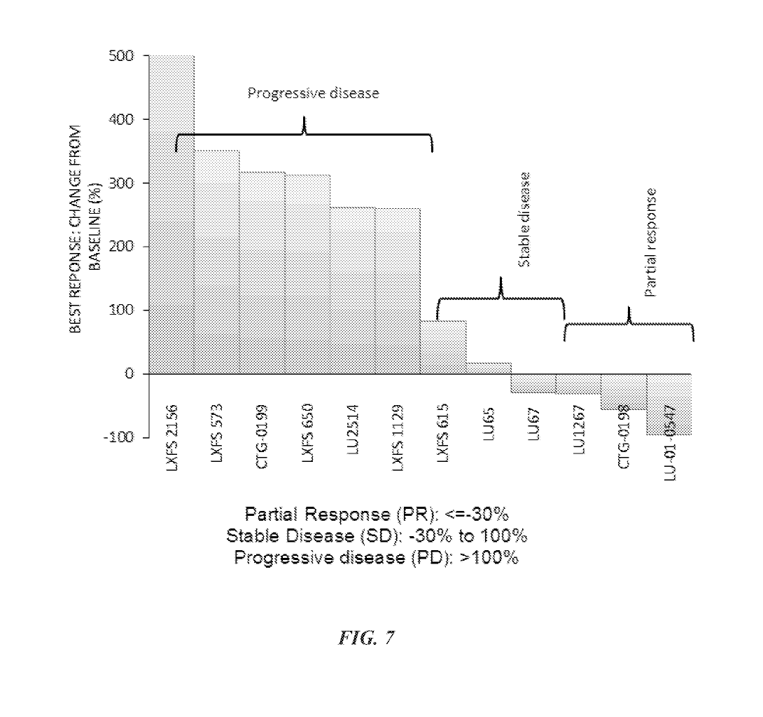

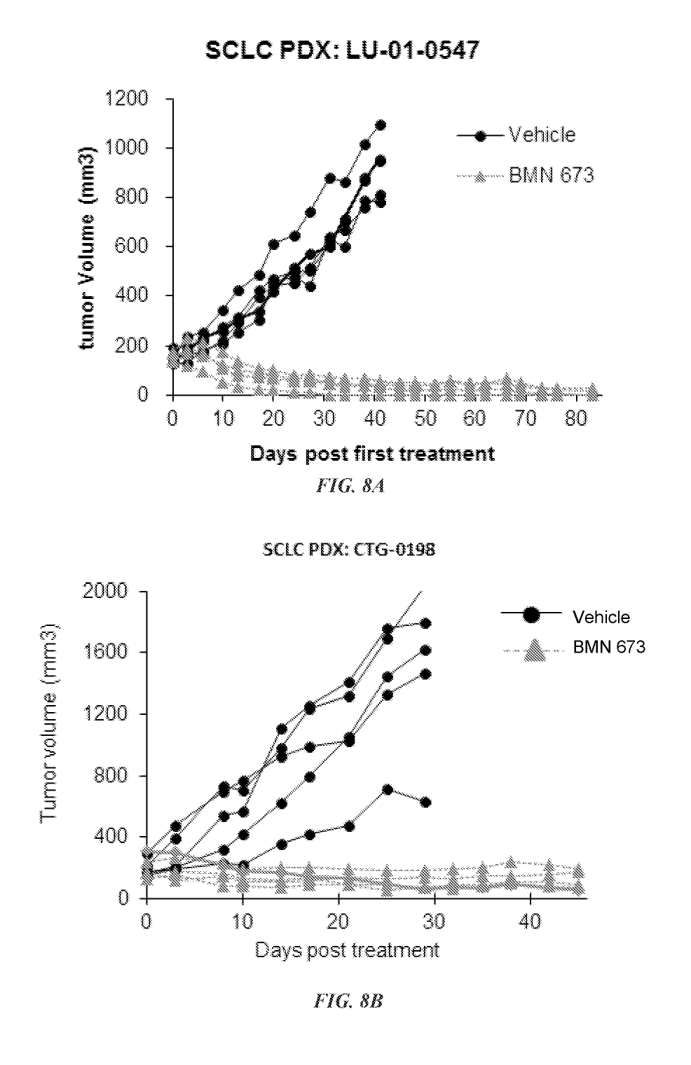

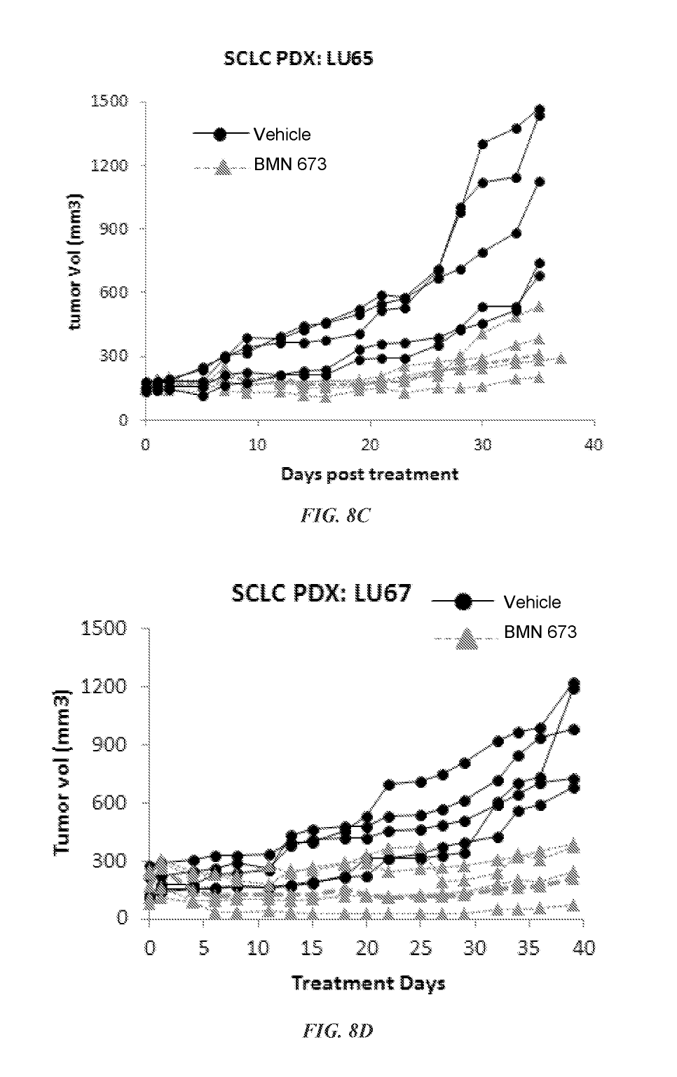

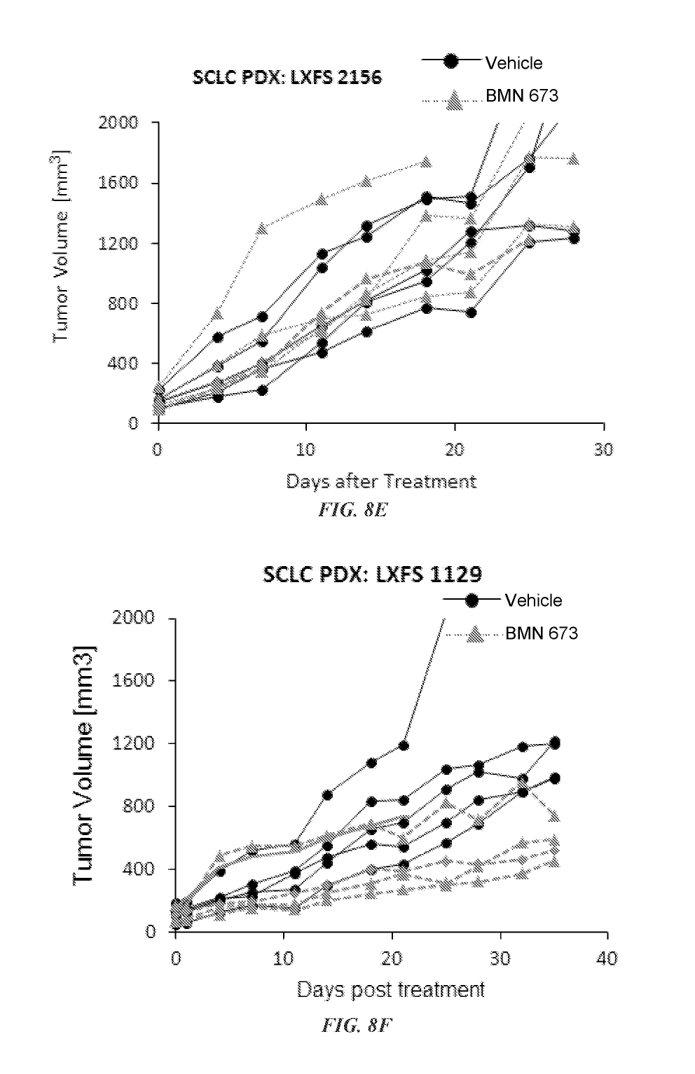

Twelve human SCLC PDX models were further tested with talazoparib at maximum tolerated doses compared to a vehicle control. Three of the 12 PDX models showed 30% or more tumor regression compared to baseline during talazoparib treatment, and were defined as partial responders (PR); three of the 12 PDX tumors exhibited stable disease (SD)-like responses with tumor growth less than 100% on Day 21 or beyond after first dosing, while the remaining PDX models were resistant to talazoparib treatment, and were designated as progressive disease (PD) (FIG. 7). Representative individual tumor growth curves are shown in FIGS. 8A-8F.

### Reverse-Phase Protein Array (RPPA)

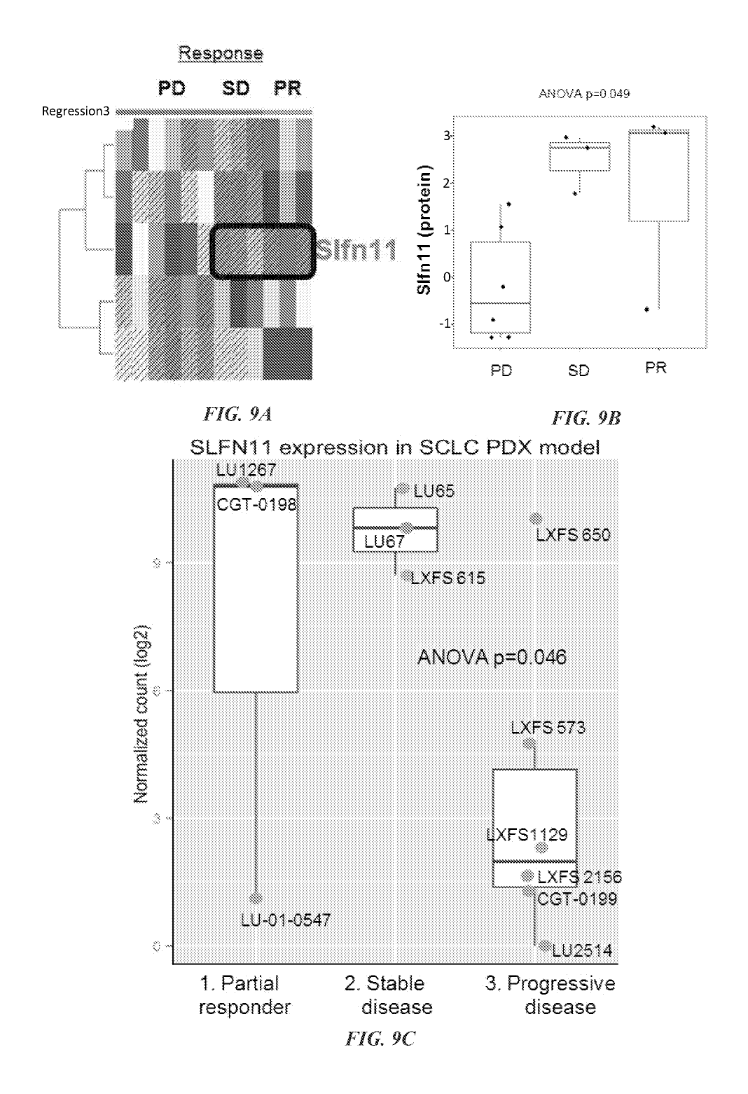

RPPA was carried out on the PDX tumor samples using the method described by Byers et al., Cancer Discovery 2012. 2, 798. The SLFN11 antibody used in the RPPA assay was obtained from Santa Cruz Biotechnology (Cat# sc-374339). RPPA analysis revealed that PR and SD response groups expressed higher average SLFN11 protein than the PD response group, with a p value of 0.049 (FIGS. 9A, 9B). At the RNA level, SLFN11 is also higher in PR and SD groups, with a p value of 0.046 (FIG. 9C).

### RNA-Seq Transcriptome Sequencing and Analysis

Two xenograft tumors were collected from each PDX model and processed to extract total RNA using AllPrep DNA/RNA Mini Kit (Qiagen). RNA samples were subjected to ribosome RNA removal using a Ribo-zero kit (Illumina) before library construction. RNA sequencing was performed with HiSeq4000 PE100. RNA-Seq paired-end reads were aligned with combined genomes from human (GRCh38, release 20 from GENCODE; see Harrow et al., Genome Res. 2012, 22(9):1760-74) and mouse (GRCm38.p3, release M4 from GENCODE) using STAR (version 2.4.1b; see Dobin et al., Bioinformatics 2012, 29(1): 15-21). The resulting alignment BAM files were sorted by read names using Samtools (version 1.2; see Li et al., Bioinformatics 2009, 25: 2078-9). The number of read pairs that were aligned to each gene in the combined human and mouse annotations (releases 20 and M4 from GENCODE, respectively) was counted by HTSeq (version 0.6.1p1; see Anders et al., Bioinformatics 2015, 31(2): 166-9). Read pairs that could be uniquely aligned to human were used to examine the relationships between gene expression and talazoparib sensitivity.

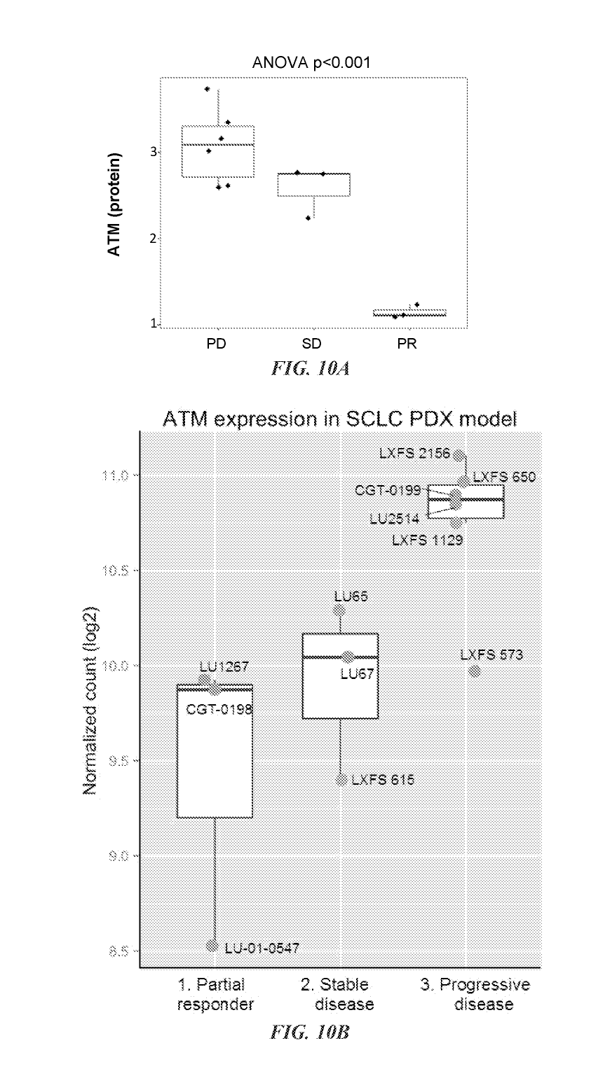

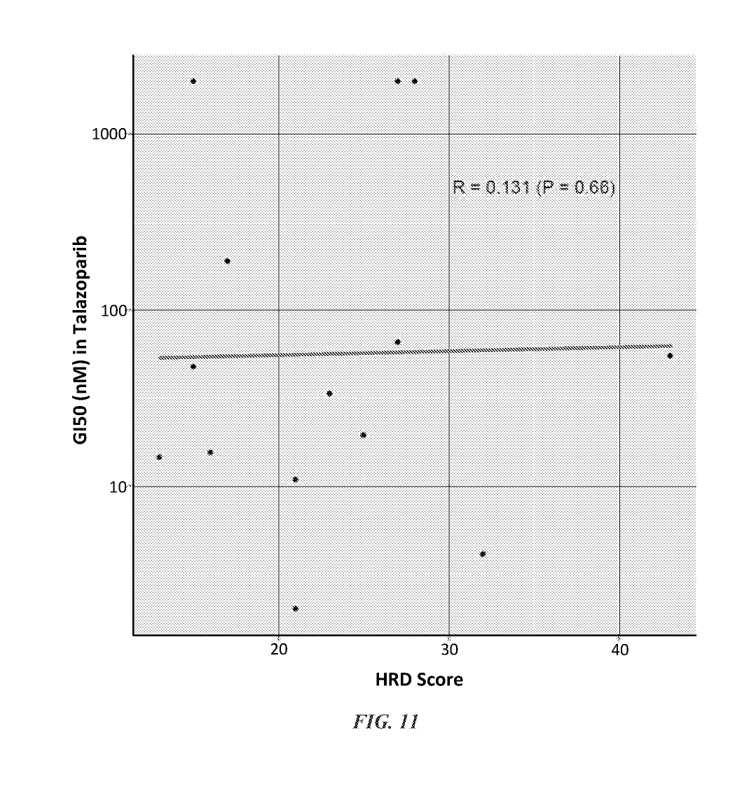

Biomarker analysis by RPPA and RNA-Seq revealed that low ATM expressing PDX models are more sensitive to talazoparib as shown in FIGS. 10A and 10B.

### Gene Mutation Analysis

Gene mutation analysis indicates that all 12 SCLC PDX tumors have TP53 or/and RB1 mutations as expected for SCLC (see Table 3).

TABLE 3

| SCLC | Prior | | | *Best response: | | Myriad |
|---|---|---|---|---|---|---|
| PDX | treat- | | | Change from base | | HRD |
| Tumor | ment | RB1 | TP53 | line (%) | | Score |
| LU-01-0547 | No | Mutation | Mutation | −95 | (D 83) | 33 |
| CTG-0198 | Yes | Wt | Mutation | −55 | (D 29) | 18 |
| LU1267 | N/A | Loss | Wt | −30 | (D 38) | 11 |
| LU67 | N/A | Mutation | Mutation | −29 | (D 25) | 29 |
| LU65 | Yes | Mutation | Wt | 17 | (D 21) | 31 |
| LXFS 615 | N/A | Loss | Mutation | 83 | (D 21) | 24 |
| LXFS 1129 | N/A | Mutation | Mutation | 260 | (D 21) | 20 |
| LU2514 | N/A | Mutation | Mutation | 262 | (D 21) | 24 |
| LXFS 650 | Yes | Mutation | Mutation | 314 | (D 21) | 17 |
| CTG-0199 | Yes | Mutation | Loss | 318 | (D 21) | 17 |
| LXFS 573 | N/A | Mutation | Mutation | 351 | (D 21) | 14 |
| LXFS 2156 | N/A | Mutation | Mutation | 615 | (D 21) | 43 |

N/A: not available.
Wt: wild type
*Median tumor volume (n = 5).

### Example 4: Comparison to Myriad HRD Score

No apparent relationship was found between talazoparib responses and the Myriad HRD (homologous recombination deficiency) score in either the SCLC cell lines tested or these SCLC PDX models as shown in FIG. 11.

All references throughout, such as publications, patents, patent applications, and published patent applications, are incorporated herein by reference in their entireties.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A method of treating small cell lung cancer in a subject expressing SLFN11, comprising administering to the subject an effective amount of a PARP inhibitor.

2. A method of treating a small cell lung cancer subject, comprising detecting one or more of SLFN11, SIL1, SLC25A3, MAF, AP3B1, C1orf50, BCL2, DDX6, or GULP1, in a tumor cell sample from the subject, and administering an effective amount of a PARP inhibitor to the subject.

3. A method of selecting a small cell lung cancer subject for PARP inhibitor chemotherapy, comprising detecting one or more of SLFN11, SIL1, SLC25A3, MAF, AP3B1, C1orf50, BCL2, DDX6, and GULP1, in a small cell lung cancer tumor sample of the subject, and administering an effective amount of a PARP inhibitor to the subject.

4. The method of claim 1, wherein the PARP inhibitor is talazoparib, olaparib, rucaparib, veliparib, CEP9722, MK4827, or BGB-290, or a pharmaceutically acceptable salt thereof.

5. The method of claim 4, wherein the PARP inhibitor is talazoparib or a pharmaceutically acceptable salt thereof.

6. The method of claim 5, wherein the PARP inhibitor is the tosylate salt of talazoparib.

7. A method of treating small cell lung cancer in a subject expressing SLFN11, comprising administering to the subject an effective amount of talazoparib or a pharmaceutically acceptable salt thereof.

8. The method of claim 1, wherein talazoparib or a pharmaceutically acceptable salt thereof is administered orally, once daily, at a dose of about 0.5 to about 2 mg per day, or of about 1 mg/day, or about 0.10 to 0.75 mg/kg/day, or about 0.25-0.30 mg/kg/day.

9. The method of claim 1, wherein the subject expresses one or more of SIL1, SLC25A3, MAF, AP3B1, C1orf50, BCL2, DDX6, or GULP1.

10. The method claim 1, wherein the subject has an increased expression level of one or more of SLFN11, SIL1, SLC25A3, MAF, AP3B1, C1orf50, BCL2, DDX6, or GULP1.

11. The method of claim 1 wherein the PARP inhibitor or talazoparib or a pharmaceutically acceptable salt thereof is administered in combination with one or more chemotherapeutic agents, surgery, and/or radiation.

12. The method of claim 11, wherein the one or more chemotherapeutic agents is a DNA damaging agent, temozolomide, a topoisomerase 1 inhibitor, irinotecan, topotecan, a topoisomerase 2 inhibitor, etoposide, enzalutamide, an ATR inhibitor, an EGFR inhibitor, a platinum drug, cisplatin, carboplatin, or etoposide.

13. The method of claim 1, wherein the subject has previously been treated with a platinum drug, or with cisplatin, or with carboplatin, optionally in combination with etoposide.

14. The method of claim 1, wherein the subject expresses a reduced level of ATM.

15. The method of claim 2, wherein one of the detected biomarkers is SLFN11.

16. The method of claim 2, wherein the detecting step comprises detection by an immunohistological assay, an immunohistochemistry staining (IHC) assay, an in-situ LC/MS assay, a promoter methylation assay, a cytological assay, an mRNA expression assay, an RT-PCR assay, a northern blot assay, a protein expression immunosorbent assay (ELISA), an enzyme-linked immunospot assay (ELISPOT), a lateral flow test assay, an enzyme immunoassay, a fluorescent polarization immunoassay, a chemiluminescent immunoassay (CLIA), or a fluorescence activated sorting assay (FACS).

17. The method of claim 1, wherein the subject expresses an increased level of one or more of SLFN11, SIL1, SLC25A3, MAF, AP3B1, C1orf50, BCL2, DDX6, or GULP1.

18. The method of claim 2, wherein the detecting step comprises detecting an increased level of one or more of SLFN11, SIL1, SLC25A3, MAF, AP3B1, C1orf50, BCL2, DDX6, or GULP1.

19. The method of claim 17, wherein the subject expresses a reduced level of ATM.

20. The method of claim 18, wherein the detecting step further comprises detecting ATM, or detecting a reduced level of expression of ATM.

21. The method of claim 1, wherein the subject expresses the TP53 and/or RB1 mutation.

22. The method of claim 1, wherein the RMA score for SLFN11 in the subject is 4 or higher, or is 5 or higher, or is 6 or higher, or is 7 or higher, or is 8 or higher.

23. The method of claim 1, wherein the subject has a Myriad HRD score of 40 or lower, or of 35 or lower, or of 30 or lower, or of 25 or lower, or of 20 or lower.
